(19) United States
(12) Patent Application Publication
(10) Pub. No.: US 2025/0277262 A1
(43) Pub. Date: Sep. 4, 2025

(54) CLICK-CHEMISTRY RETENTION OF FLUORESCENT NUCLEOTIDES

(71) Applicant: 10x Genomics, Inc., Pleasanton, CA (US)

(72) Inventors: Anton Jan BJÖRNINEN, Stockholm (SE); Debashish CHITNIS, Stocklholm (SE); Toon VERHEYEN, Solna (SE)

(21) Appl. No.: 19/046,288

(22) Filed: Feb. 5, 2025

Related U.S. Application Data
(60) Provisional application No. 63/550,440, filed on Feb. 6, 2024.

(51) Int. Cl. C12Q 1/6869; C12Q 1/6806

(52) U.S. Cl. CPC C12Q 1/6869; C12Q 1/6806

(57) ABSTRACT

The present disclosure relates in some aspects to methods, systems, and kits for sequencing a template nucleic acid molecule using click chemistry bioconjugation. In some aspects, a coupling reaction is performed between a first functional group of a modified nucleotide molecule and a second functional group of a modified 3′ reversibly terminated nucleotide on a priming strand. In some aspects, this coupling reaction creates stable retention of the fluorescent read-out.

### CROSS-REFERENCE TO RELATED APPLICATIONS

This application claims the priority benefit of U.S. Provisional Patent Application Ser. No. 63/550,440, filed Feb. 6, 2024, the contents of which are incorporated herein by reference in their entirety.

### REFERENCE TO AN ELECTRONIC SEQUENCE LISTING

The contents of the electronic sequence listing (202412021500SEQLIST.xml; Size: 9,814 bytes; and Date of Creation: May 12, 2025) is herein incorporated by reference in its entirety.

### FIELD

The present disclosure relates in some aspects to methods for sequencing nucleic acid molecules, including methods for in situ sequencing and analysis of target nucleic acids in a biological sample.

### BACKGROUND

Nucleic acid sequencing is a versatile tool that helps scientists advance the understanding of biology and has wide-ranging applications in various fields, such as medical diagnostics, biotechnology, forensic biology, and virology. Currently, there are several sequencing methods available, including Maxam-Gilbert sequencing, Sanger (chain-termination) sequencing, and next-generation sequencing (NGS) techniques. NGS sequencing-by-synthesis (SBS) is based on incorporation of a fluorescent, reversibly terminated nucleotide into an extended priming strand, where the incorporated nucleotide is complementary to a nucleotide in the template nucleic acid molecule that is being probed. Both the reversible terminator and the fluorescent moiety must be cleaved off the newly incorporated nucleotide before progressing to the next cycle of sequencing, leaving a “molecular scar” that destabilizes the extended priming strand and limit the length of template sequence that can be sequenced. Thus, improved methods for sequencing nucleic acid molecules that simplify the steps required, improve signal detection and/or that minimize the “molecular scarring” phenomenon are of interest in the field.

Recently, new sequencing technologies have emerged that attempt to improve signal detection while circumventing the “molecular scarring” phenomenon by incorporating a reversible terminator at the 3′ terminus of the priming strand and performing stochastic binding of a polymerase and fluorescent nucleotide to form a bound complex. As this fluorescent nucleotide is not incorporated into the priming strand (but can still be detected as part of the bound complex), it does not leave a molecular scar and thus enables the ability to sequence longer template sequences in a single sequence read. As the formation of the bound complex is driven by stochastic binding, complex formation is primarily driven by local concentrations of the polymerase and nucleotides, thereby leading to an inherent instability of the complex upon rinsing away unbound polymerase and fluorescent nucleotides. Such stochastic binding-based methods are promising; however, there is a need in the field for methods that increase the stability of the transiently formed complex.

### SUMMARY

The present disclosure provides improved methods for sequencing nucleic acid molecules that include conjugating the complementary fluorescent nucleotide in the bound complex to the reversibly terminated nucleotide on 3′ end of the priming stand (e.g., via click chemistry bioconjugation) in order to retain the fluorescent nucleotide after the polymerase-nucleotide mixture is removed. The present disclosure, in some aspects, provides methods, kits, and systems for sequencing analysis of a template nucleic acid molecule where a coupling reaction is performed between a first functional group of a modified nucleotide molecule and a second functional group of a modified 3′ reversibly terminated nucleotide on a priming strand. In any of the embodiments described herein, this coupling reaction creates stable retention of the fluorescent read-out to improve signal detection, minimize “molecular scarring” and potentially enable longer sequence reads.

In some embodiments, provided herein is a method for sequencing a template nucleic acid molecule comprising: a) contacting a priming strand bound to the template nucleic acid molecule with (i) a polymerase and (ii) a first plurality of modified nucleotide molecules that each comprise a first functional group, to form a complex comprising a 3′ terminus of the priming strand, the template nucleic acid molecule, the polymerase, and a first modified nucleotide molecule of the first plurality of modified nucleotide molecules, wherein the priming strand comprises a modified 3′ reversibly terminated nucleotide that comprises a second functional group, and wherein the first modified nucleotide molecule is not incorporated into a sugar-phosphate backbone of the priming strand; b) performing a coupling reaction between the first functional group and the second functional group to covalently couple the first modified nucleotide molecule included in the complex to the modified 3′ reversibly terminated nucleotide; and c) detecting a presence of the first modified nucleotide molecule coupled to the modified 3′ reversibly terminated nucleotide to identify a complementary nucleotide in the template nucleic acid molecule.

In some embodiments, provided herein is a method for sequencing a template nucleic acid molecule comprising: providing: i) one or more reagents comprising a polymerase and a first plurality of modified nucleotide molecules, each comprising a first functional group, and ii) a priming strand bound to the template nucleic acid molecule, wherein the priming strand comprises a modified 3′reversibly terminated nucleotide comprising a second functional group; a) contacting the priming strand bound to the template nucleic acid molecule with the one or more reagents, to form a complex comprising a 3′ terminus of the priming strand, the template nucleic acid molecule, the polymerase, and a first modified nucleotide molecule of the first plurality of modified nucleotide molecules, wherein the first modified nucleotide molecule is not incorporated into a sugar-phosphate backbone of the priming strand; b) performing a coupling reaction between the first functional group and the second functional group to covalently couple the first modified nucleotide molecule included in the complex to the modified 3′ reversibly terminated nucleotide; and c) detecting a presence of the first modified nucleotide molecule coupled to the modified 3′ reversibly terminated nucleotide to identify a complementary nucleotide in the template nucleic acid molecule.

In any of the embodiments herein, the coupling reaction can comprise a click chemistry reaction. In any of the embodiments herein, the first functional group of each of the first plurality of modified nucleotides can be attached to the modified nucleotide using a first linker. In any of the embodiments herein, the first linker can comprise a polyethylene glycol (PEG) linker. In any of the embodiments herein, the polyethylene glycol (PEG) linker can comprise 2 to 24 ethylene glycol units. In any of the embodiments herein, the first linker can comprise a cleavable linker. In any of the embodiments herein, the cleavable linker can comprise a photocleavable linker, a Pd-cleavable linker, a phosphine-cleavable linker, an ester linker, or a disulfide linker.

In any of the embodiments herein, the second functional group can be attached to the modified 3′ reversibly terminated nucleotide using a second linker. In any of the embodiments herein, the second linker can comprise a cleavable linker. In any of the embodiments herein, the cleavable linker can comprise a photocleavable linker, a Pd-cleavable linker, a phosphine-cleavable linker, an ester linker, or a disulfide linker. In any of the embodiments herein, the second linker and the first linker can be the same.

In any of the embodiments herein, the first functional group is attached directly or indirectly to the nucleobase of each of the plurality of modified nucleotide molecules. In any of the embodiments herein, at least a subset of the plurality of modified nucleotide molecules each comprise a purine nucleobase, and the first functional group is attached directly or indirectly to the C7 position of the nucleobase of each of the plurality of modified nucleotide molecules. In any of the embodiments herein, at least a subset of the plurality of modified nucleotide molecules each comprise a pyrimidine nucleobase, and the first functional group is attached directly or indirectly to the C5 position of the nucleobase of each of the plurality of modified nucleotide molecules. In any of the embodiments herein, the first functional group is attached directly or indirectly to the sugar of each of the plurality of modified nucleotide molecules, optionally wherein the first functional group is attached directly or indirectly to 3′ position or 5′ position of the sugar. In any of the embodiments herein, the second functional group is attached directly or indirectly to the nucleobase of the modified 3′ reversibly terminated nucleotide. In any of the embodiments herein, the nucleobase is a purine, and the second functional group is attached directly or indirectly to the C7 position of the nucleobase. In any of the embodiments herein, the nucleobase is a pyrimidine, and the second functional group is attached directly or indirectly to the C5 position of the nucleobase. In any of the embodiments herein, the second functional group is attached directly or indirectly to the sugar of the modified 3′ reversibly terminated nucleotide, optionally wherein the second functional group is attached directly or indirectly to 3′ position or 5′ position of the sugar.

In any of the embodiments herein, the first functional group and second functional group can be selected from a list of click reactive functional group pairs comprising azido/alkynyl groups, alkynyl/azido groups, azido/dibenzocyclooctynyl (DBCO) groups, dibenzocyclooctynyl (DBCO)/azido groups, azido/cyclooctynyl groups, cyclooctynyl/azido groups, tetrazine/dienophile groups, dienophile/tetrazine groups, thiol/alkynyl groups, alkynyl/thiol groups, cyano/1,2-amino thiol groups, 1,2-amino thiol/cyano groups, nitrone/cyclooctynyl groups, cyclooctynyl/nitrone groups, or any combination thereof.

In any of the embodiments herein, the first functional group can comprise an azido group and the second functional group can comprise a dibenzocyclooctynyl (DBCO) group. In any of the embodiments herein, the azido group can be attached directly or indirectly to a nucleobase in the first plurality of modified nucleotide molecules. In any of the embodiments herein, the azido group can be attached directly or indirectly to a 3′ position of a sugar in the first plurality of modified nucleotide molecules. In any of the embodiments herein, the azido group can be attached directly or indirectly to a 5′ position of a sugar in the first plurality of modified nucleotide molecules. In any of the embodiments herein, the dibenzocyclooctynyl (DBCO) group can be attached directly or indirectly to a base in the modified 3′ reversibly terminated nucleotide. In any of the embodiments herein, the dibenzocyclooctynyl (DBCO) group can be attached directly or indirectly to a 3′ position of a sugar in the modified 3′ reversibly terminated nucleotide. In any of the embodiments herein, the dibenzocyclooctynyl (DBCO) group can be attached directly or indirectly to a 5′ position of a sugar in the modified 3′ reversibly terminated nucleotide. In any of the embodiments herein, the dibenzocyclooctynyl (DBCO) group can be attached directly or indirectly to a reversible blocking group of the modified 3′ reversibly terminated nucleotide.

In any of the embodiments herein, the first functional group can comprise a dibenzocyclooctynyl (DBCO) group and the second functional group can comprise an azido group. In any of the embodiments herein, the dibenzocyclooctynyl (DBCO) group can be attached directly or indirectly to a base in the first plurality of modified nucleotide molecules. In any of the embodiments herein, the dibenzocyclooctynyl (DBCO) group can be attached directly or indirectly to a 3′ position of a sugar in the first plurality of modified nucleotide molecules. In any of the embodiments herein, the dibenzocyclooctynyl (DBCO) group can be attached directly or indirectly to a 5′ position of a sugar in the first plurality of modified nucleotide molecules. In any of the embodiments herein, the azido group can be attached directly or indirectly to a base in the modified 3′ reversibly terminated nucleotide. In any of the embodiments herein, the azido group can be attached directly or indirectly to a 3′ position of a sugar in the modified 3′ reversibly terminated nucleotide. In any of the embodiments herein, the azido group can be attached directly or indirectly to a 5′ position of a sugar in the modified 3′ reversibly terminated nucleotide. In any of the embodiments herein, the azido group can be attached directly or indirectly to a reversible blocking group of the modified 3′ reversibly terminated nucleotide.

In any of the embodiments herein, the modified 3′ reversibly terminated nucleotide can be a modified 3′-O-blocked reversibly terminated nucleotide. In any of the embodiments herein, the modified 3′-O-blocked reversibly terminated nucleotide can be a modified 3′-O-azidomethyl deoxynucleotide triphosphate (3′-O-azidomethyl dNTP), a modified 3′-O-allyl deoxynucleotide triphosphate (3′-O-allyl-dNTP), or a modified 3′-O-amino deoxynucleotide triphosphate (3′-O-NH₂ dNTP). In any of the embodiments herein, the modified 3′ reversibly terminated nucleotide can be a modified 3′-unblocked reversibly terminated nucleotide.

In any of the embodiments herein, the method can further comprise: d) performing a cleavage reaction to remove the covalently coupled modified nucleotide molecule from the modified 3′ reversibly terminated nucleotide; c) performing a deprotection reaction to deprotect the modified 3′ reversibly terminated nucleotide; and f) performing an extension reaction to incorporate a modified 3′ reversibly terminated nucleotide comprising the second functional group that is complementary to the identified nucleotide in the template nucleic acid molecule into an extended priming strand. In any of the embodiments herein, the cleavage reaction and the deprotection reaction can be the same reaction.

In any of the embodiments herein, the method can further comprise performing a first wash step to remove unbound polymerase and unbound modified nucleotide molecules prior to performing the detecting step. In any of the embodiments herein, the method can further comprise performing a second wash step after performing the detecting step to disrupt the complex.

In any of the embodiments herein, the second functional group is attached indirectly to 3′ reversibly terminated molecule via a cleavable linker, and d) performing the cleavage reaction comprises contacting the complex with a reagent capable of cleaving the cleavable linker. In any of the embodiments herein, the cleavable linker comprises a disulfide linker, and the reagent comprises a disulfide reducing agent, optionally wherein the disulfide reducing agent comprises Dithiothreitol (DTT).

In any of the embodiments herein, the method can further comprise repeating steps (a)-(f) for at least one additional cycle using at least one additional plurality of modified nucleotide molecules that comprise the first functional group to detect a presence of a modified nucleotide molecule coupled to the modified 3′ reversibly terminated nucleotide and identify at least one additional complementary nucleotide in the template nucleic acid molecule. In any of the embodiments herein, the at least one additional cycle can comprise at least 2, 5, 10, 20, 30, 40, or 50 additional cycles.

In any of the embodiments herein, the method can further comprise: prior to performing a first contacting step in (a), hybridizing a primer that does not comprise a modified 3′ reversibly terminated nucleotide at its 3′ end to a primer binding site in the template nucleic acid molecule; and performing an extension reaction to incorporate a modified 3′ reversibly terminated nucleotide that comprises the second functional group and that is complementary to a nucleotide in the template nucleic acid molecule into an extended primer strand to generate the priming strand.

In any of the embodiments herein, the first plurality of modified nucleotide molecules and the at least one additional plurality of modified nucleotide molecules can comprise a same set of modified nucleotide molecules. In any of the embodiments herein, the first plurality of modified nucleotide molecules and the at least one additional plurality of modified nucleotide molecules can comprise different sets of modified nucleotide molecules. In any of the embodiments herein, the first plurality of modified nucleotide molecules and the at least one additional plurality of modified nucleotide molecules can each comprise modified nucleotide molecules that do not include a 3′ reversible terminator moiety. In any of the embodiments herein, the first plurality of modified nucleotide molecules and the at least one additional plurality of modified nucleotide molecules can each comprise one or more modified nucleotide molecules that are coupled to a detectable label. In any of the embodiments herein, modified nucleotide molecules that are coupled to a detectable label and that comprise a different nucleobase can be coupled to a different detectable label. In any of the embodiments herein, the first plurality of modified nucleotide molecules and the at least one additional plurality of modified nucleotide molecules can each comprise at least one modified nucleotide molecule that is not labeled with a detectable label.

In any of the embodiments herein, detecting a presence of a modified nucleotide molecule in the complex can comprise detecting a signal associated with a detectable label coupled to the modified nucleotide molecule. In any of the embodiments herein, detecting a presence of a modified nucleotide molecule in the complex can comprise detecting an absence of a signal, wherein the absence of signal is associated with a modified nucleotide molecule that is not coupled to a detectable label. In any of the embodiments herein, the polymerase can be not labeled with a detectable label. In any of the embodiments herein, the detectable label can be a fluorophore. In any of the embodiments herein, the first plurality of modified nucleotide molecules can comprise a set of four different modified nucleotide molecules, and wherein each different modified nucleotide molecule is coupled to a different fluorophore. In any of the embodiments herein, the first plurality of modified nucleotide molecules can comprise a set of four different modified nucleotide molecules, and wherein three of the four different modified nucleotide molecules are coupled to different fluorophores and one of the four different modified nucleotide molecules is not conjugated to a fluorophore. In any of the embodiments herein, the first plurality of modified nucleotide molecules can comprise a set of four different modified nucleotide molecules, and wherein two of the four different modified nucleotides are coupled to different fluorophores, one of the four different modified nucleotide molecules is coupled to the two different fluorophores, and one of the four different modified nucleotide molecules is not conjugated to a fluorophore.

In any of the embodiments herein, the first plurality of modified nucleotides and the at least one additional plurality of modified nucleotides can be selected from modified A, T, U, C, and G. In any of the embodiments herein, the first plurality of modified nucleotides and the at least one additional plurality of modified nucleotides can be selected from modified A, T, C, and G. In any of the embodiments herein, the template nucleic acid molecule can comprise a DNA molecule. In any of the embodiments herein, the template nucleic acid molecule can comprise an RNA molecule. In any of the embodiments herein, the complex can comprise a transient complex. In any of the embodiments herein, the transient complex persists for at least 5 sec, 10 sec, 20 sec, 30 sec, 40 sec, 50 sec, 1 min, 2 min, 3 min, 4 min, 5 min, or 10 min.

In any of the embodiments herein, the template nucleic acid molecule can comprise a target analyte nucleic acid molecule. In any of the embodiments herein, the template nucleic acid molecule can comprise a barcode sequence associated with a target analyte. In any of the embodiments herein, the method can further comprise hybridizing a circularizable probe to the target analyte or to a labeling agent bound to the target analyte and ligating the circularizable probe to form a circularized probe, wherein the method further comprises performing rolling circle amplification of the circularized probe to generate the template nucleic acid molecule. In any of the embodiments herein, the circularizable probe can be a padlock probe sequence. In any of the embodiments herein, the target analyte nucleic acid molecule can comprise an mRNA molecule.

In any of the embodiments herein, the template nucleic acid molecule to be sequenced can be attached to a solid support. In any of the embodiments herein, the solid support can comprise a sequencing flow cell. In any of the embodiments herein, the template nucleic acid molecule can be sequenced in situ in a cell sample or tissue sample. In any of the embodiments herein, the cell sample can comprise a layer of cells deposited on a surface.

In some embodiments, provided herein is a kit for sequencing a template nucleic acid molecule comprising: a plurality of modified nucleotide molecules that each comprise a first functional group; a primer designed to hybridize to the template nucleic acid molecule, wherein the primer comprises a modified 3′ reversibly terminated nucleotide that comprises a second functional group that is reactive with the first functional group; a modified 3′ reversibly terminated nucleotide that comprises the second functional group; a polymerase; or any combination thereof. In any of the embodiments herein, the plurality of modified nucleotide molecules can comprise a set of four modified nucleotide molecules, wherein each of the four modified nucleotide molecules comprises a different nucleobase and a different detectable feature, wherein the detectable feature is selected from: (i) a fluorophore that is different from fluorophores coupled to other modified nucleotide molecules in the set, and (ii) optionally, an absence of a fluorophore. In any of the embodiments herein, the first functional group can comprise an azido group and the second functional group can comprise a dibenzocyclooctynyl (DBCO) group.

In some aspects, provided herein is a system comprising: one or more processors; and a memory communicatively coupled to the one or more processors and configured to store instructions that, when executed by the one or more processors, cause the system to perform the method of any of the preceding embodiments.

### BRIEF DESCRIPTION OF THE DRAWINGS

The drawings illustrate certain features and advantages of this disclosure. These embodiments are not intended to limit the scope of the appended claims in any manner.

FIG. 1 provides a non-limiting example of a process flowchart for sequencing a template nucleic acid molecule in accordance with one implementation of the methods described herein.

FIG. 2 provides a non-limiting example of a process flowchart for sequencing a template nucleic acid molecule in accordance with one implementation of the methods described herein.

FIG. 3 depicts a system for performing a sequencing assay, in accordance with some implementations of the methods described herein.

FIG. 4 depicts a computer system or computer network, in accordance with some instances of the systems described herein.

FIG. 5 provides a schematic illustration of a method for sequencing a template nucleic acid molecule in accordance with one implementation of the methods described herein.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Methods for sequencing nucleic acid molecules are described that are based on conjugating a fluorescently-labeled modified nucleotide (comprising a first functional group) to a reversibly terminated modified nucleotide (comprising a second functional group) positioned at the 3′ end of a priming stand (using, e.g., click chemistry bioconjugation) when a complementary fluorescently-labeled modified nucleotide is present in a complex comprising the 3′ terminus of the priming strand, the template nucleic acid molecule to which it is bound, and a polymerase, in order to retain the fluorescent nucleotide after the polymerase-nucleotide mixture used to contact the primed template nucleic acid is removed. The disclosed methods, compositions, kits, and systems for performing sequencing of a template nucleic acid molecule are applicable to various multi-cycle sequencing approaches, including in situ sequencing and flow cell sequencing. The coupling reaction performed between a first functional group of a modified nucleotide molecule and a second functional group of a modified 3′ reversibly terminated nucleotide on a priming strand bound to a template nucleic acid creates stable retention of the fluorescent read-out to improve signal detection, minimize “molecular scarring” and potentially enable longer sequence reads.

Additional aspects of the methods, compositions, kits, and systems disclosed herein are described in the sections below.

### II. Nucleic Acid Sequencing Using Click Chemistry Retention of Fluorescent Nucleotides

The click chemistry-based sequencing methods described herein are useful for multi-cycle sequencing approaches where nucleotides of nucleotide strand are “interrogated” by binding to a complementary nucleotide. For example, the click chemistry-based sequencing methods described herein are applicable to both in situ sequencing applications (e.g., in situ sequencing of endogenous nucleic acid sequences and/or target-specific barcode sequences associated with target analytes of interest that are distributed within a cell or tissue sample) and to more conventional “sequencing in a flow cell” applications (e.g., sequencing of endogenous nucleic acid sequences extracted from a cell or tissue sample). The in situ and flow cell sequencing approaches differ in terms of the sample preparation steps required, as described elsewhere herein, but can share common features in terms of the cyclic series of steps performed to identify nucleotides base-by-base in a template nucleic acid sequence (e.g., a target analyte sequence and/or an associated target-specific barcode sequence).

In some instances, the click chemistry-based sequencing methods described herein comprise contacting a template nucleic acid molecule with a sequencing primer designed to hybridize to a portion of the template nucleic acid molecule, where the sequencing primer includes a modified, reversibly terminated nucleotide at its 3′ end that includes a second functional group (e.g., a second click chemistry reactive group). The modified, 3′ reversibly terminated nucleotide blocks incorporation of nucleotides into the sugar-phosphate backbone of the priming strand when contacting primed template nucleic acid molecules with a polymerase and a plurality of nucleotides (e.g., a plurality of modified nucleotides that comprise a complementary first functional group (e.g., a first click chemistry reactive group that is reactive with the second click chemistry reactive group attached to the modified, 3′ reversibly terminated nucleotide). In some instances, the sequencing primer may comprise a free 3′-hydroxyl group at its 3′ terminus, and an initial primer extension reaction may be performed to incorporate a modified, 3′ reversibly terminated nucleotide that includes the second functional group at the 3′ terminus of the bound primer (i.e., the 3′ terminus of the priming strand).

In some instances, the click chemistry-based sequencing methods described herein may comprise performing a cyclic series of base-by-base sequencing reactions, where each sequencing cycle comprises:
(i) contacting the priming strand bound to the template nucleic acid molecule with a polymerase and a modified nucleotide (e.g., at least one modified nucleotide or a plurality of modified nucleotides) comprising the first functional group to form a complex comprising the 3′ terminus of the priming strand, the template nucleic acid molecule, the polymerase, and a modified nucleotide comprising the first functional group that is complementary to a nucleotide in the template nucleic acid molecule, where the modified nucleotide comprising the first functional group of the complex is not incorporated into the priming strand (i.e., it is not incorporated into the sugar-phosphate backbone of the priming strand) because of the presence of 3′ reversibly terminated nucleotide. In some instances, the at least one modified nucleotide (or at least one modified nucleotide of the plurality of modified nucleotides) may further comprise a detectable label (e.g., a fluorescent label) in addition to the first functional group.
(ii) performing a coupling reaction between the first functional group and the second functional group to covalently couple the modified nucleotide molecule included in the complex to the modified 3′ reversibly terminated nucleotide of the priming strand; and
(iii) detecting the presence of the modified nucleotide molecule coupled to the modified 3′ reversibly terminated nucleotide of the priming strand to identify a complementary nucleotide in the template nucleic acid molecule. In some instances, detecting the presence of the modified nucleotide molecule may comprise detecting a signal (e.g., a fluorescence signal) associated with a detectably-labeled modified nucleotide molecule (e.g., a fluorescently-labeled modified nucleotide molecule). In some instances, detecting the presence of the modified nucleotide molecule may comprise detecting an absence of signal (e.g., the modified nucleotide that is complementary to the nucleotide in the template nucleic acid molecule may not comprise a fluorophore or other detectable label).

### Template Nucleic Acid Molecules

In some instances, the template nucleic acid molecule includes a target analyte nucleic acid molecule (e.g., a DNA molecule, an RNA molecule, or an mRNA molecule). In some instances, the template nucleic acid includes a reporter oligonucleotide, such as a barcode.

In some instances, the template nucleic acid molecule is a DNA molecule. Examples of DNA template nucleic acid molecules include DNA molecules such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA molecule can copies from another nucleic acid molecule (e.g., DNA or RNA such as mRNA).

In some instances, the template nucleic acid molecule is an RNA molecule. Examples of RNA template nucleic acid molecules include RNA molecules such as various types of coding and non-coding RNA. Examples of the different types of RNA molecules include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at 3′ end), and a spliced mRNA in which one or more introns have been removed. Also included in the analytes disclosed herein are non-capped mRNA, a non-polyadenylated mRNA, and a non-spliced mRNA. The RNA template nucleic acid molecule can be copied from another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, IRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some aspects, the template nucleic acid comprises a nucleic acid analyte derived from a biological sample and/or a reporter oligonucleotide or nucleic acid marker associated with an analyte from a biological sample. Such analytes can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

In some instances, a template nucleic acid can include a reporter oligonucleotide or marker associated with the presence of an analyte (e.g., an endogenous analyte) in a sample. Such analytes may include nucleic acid analytes and/or non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Examples of analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

In some instances, a template nucleic acid molecule may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

Methods and compositions disclosed herein can be used to analyze any number of template nucleic acid molecules (e.g., nucleic acid analytes and/or analyte-associated barcode sequences) or fragments thereof. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of a sample (e.g., a cell sample or tissue sample) or tethered within individual features on a substrate (e.g., a flow cell surface).

### Modified Nucleotides

In some instances, each cycle of a cyclic series of base-by-base sequencing reactions performed as part of the disclosed methods for sequencing (e.g., in situ or flow cell sequencing) may comprise contacting priming strands bound to template nucleic acid molecules with at least one modified nucleotide molecule comprising the first functional group (e.g., a click chemistry reactive group, as described elsewhere herein). In some instances, each cycle of a cyclic series of base-by-base sequencing reactions may comprise contacting priming strand bound to a template nucleic acid molecule with a plurality of modified nucleotide molecules (e.g., 2, 3, or 4 modified nucleotide molecules) comprising the first functional group.

Such modified nucleotide molecules may be modified versions of any of a variety of naturally-occurring nucleotides and/or functional analogs thereof (e.g., nucleotide analogs capable of hybridizing to a nucleic acid sequence in a sequence-specific/correctly base-paired manner) to probe a template nucleic acid sequence. Naturally-occurring nucleotides include deoxyribonucleotides (found in DNA) that comprise a deoxyribose sugar moiety, and ribonucleotides (found in RNA) that comprise a ribose sugar moiety. Naturally-occurring deoxyribonucleotides comprise a nucleobase (or “base”) selected from the group consisting of adenine (A), thymine (T), cytosine (C), or guanine (G). Naturally-occurring ribonucleotides comprise a nucleobase selected from the group consisting of uracil (U), adenine (A), cytosine (C), or guanine (G). In some instances, the nucleotides may be terminated (e.g., reversibly terminated). In some instances, the nucleotides may be conjugated to a detectable label, e.g., a fluorophore. In some instances, as noted above, the nucleotides may be conjugated to other moieties, e.g., reactive functional groups.

In some instances, the set of one or more modified nucleotide molecules contacted with the primed template nucleic acid molecule(s) in each cycle of a multicycle sequencing process may comprise modified versions of A, T, U, C, and/or G. In some instances, the set of one or more modified nucleotide molecules contacted with the primed template nucleic acid molecule(s) in each cycle of a multicycle sequencing process may comprise modified versions of A, T, C, and/or G.

In some instances, the set of one or more modified nucleotide molecules contacted with the primed template nucleic acid molecule(s) may be the same in each cycle of a multicycle sequencing process (e.g., the one or more modified nucleotide molecules may comprise the same selection of modified A, T, U, C, and/or G in each cycle). In some instances, the set of one or more modified nucleotide molecules contacted with the primed template nucleic acid molecule(s) may be different in different cycles of a multicycle sequencing process (e.g., the one or more modified nucleotide molecules may comprise a different selection of modified A, T, U, C, and/or G in different cycles).

In some instances, the set of one or more modified nucleotide molecules contacted with the primed template nucleic acid molecule(s) may be coupled to one or more detectable labels, where different modified nucleotide molecules (e.g., modified nucleotides comprising different nucleobases) are labeled with different detectable labels. In some instances, the one or more detectable label(s) may comprise one or more fluorophores. Examples of suitable detectable labels, e.g., fluorophores, are described elsewhere herein.

In some instances, all of the modified nucleotide molecules in the set of one or more modified nucleotide molecules (e.g., modified versions of A, T, U, C, and/or G) contacted with the primed template nucleic acid molecule(s) may be coupled to detectable labels (e.g., fluorophores). In some instances, at least one modified nucleotide molecule of the set of one or more modified nucleotide molecules contacted with the primed template nucleic acid molecules may not be coupled to a detectable label. In some instances, non-labeled modified nucleotide molecules may be used to implement different detection schemes (e.g., two color, three color, or four color detection schemes). In some instances, non-labeled modified nucleotide molecules may be used to implement the readout of target-specific barcode designs used to minimize optical crowding when performing in situ sequencing (see, e.g., PCT International Patent Application Publication Nos. WO 2022/060889 and WO 2023/220300, each of which is incorporated herein by reference in its entirety).

In some instances, for example, the set of one or more modified nucleotide molecules may comprise a set of four different modified nucleotide molecules (e.g., selected from modified versions of A, T, U, C, and/or G), where each different modified nucleotide molecule (comprising a different nucleobase) is coupled to a different fluorophore.

In some instances, the set of one or more modified nucleotide molecules may comprise a set of four different modified nucleotide molecules (e.g., selected from modified versions of A, T, U, C, and/or G), where three of the four different modified nucleotide molecules (comprising different nucleobases) are coupled to different fluorophores and one of the four different modified nucleotide molecules is not conjugated to a fluorophore.

In some instances, the set of one or more modified nucleotide molecules may comprise a set of four different modified nucleotide molecules (e.g., selected from modified versions of A, T, U, C, and/or G), where two of the four different modified nucleotides (comprising different nucleobases) are coupled to different fluorophores, one of the four different modified nucleotide molecules is coupled to both of the two different fluorophores, and one of the four different modified nucleotide molecules is not conjugated to a fluorophore.

### Modified Reversible Terminators

The modified, reversibly terminated nucleotide comprising a modified, reversibly terminated nucleotide at its 3′ end that includes a second functional group (e.g., a second click chemistry reactive group, as described elsewhere herein) may comprise a modified version of any of a variety of reversibly terminated nucleotides known to those of skill in the art.

In some instances, for example, the modified 3′ reversibly terminated nucleotide may be a modified 3′-O-blocked reversibly terminated nucleotide. In some instances, the modified 3′-O-blocked reversibly terminated nucleotide may be, e.g., a modified 3′-O-azidomethyl deoxynucleotide triphosphate (3′-O-azidomethyl dNTP), a modified 3′-O-allyl deoxynucleotide triphosphate (3′-O-allyl-dNTP), or a modified 3′-O-amino deoxynucleotide triphosphate (3′-O-NH₂ dNTP). In some instances, the modified 3′ reversibly terminated nucleotide may be a modified 3′-unblocked reversibly terminated nucleotide.

### Click Chemistry Bioconjugation

In some instances, the disclosed methods comprise performing a coupling reaction between the first functional group and the second functional group to covalently couple the first modified nucleotide molecule included in the complex to the modified 3′ reversibly terminated nucleotide. In some instances, the coupling reaction is a click chemistry reaction. Any suitable click chemistry is contemplated for use with the compositions, systems, and methods described herein, for example see, e.g., Gartner and Liu (2001), Journal of the American Chemical Society 123 (28): 6961-6963; Seckute et al. (2013), Nucleic Acids Research 41 (15): e148; and Patterson et al. (2014), ACS Chem. Biol. 9 (3): 592-605, all of which are incorporated by reference herein in their entirety.

In some instances, the first functional group can be attached directly or indirectly to the nucleobase of each of the plurality of modified nucleotide molecules. In some instances, for example, at least a subset of the plurality of modified nucleotide molecules each comprise a purine nucleobase, and the first functional group is attached directly or indirectly to the C7 position of the nucleobase of each of the plurality of modified nucleotide molecules. In some instances, at least a subset of the plurality of modified nucleotide molecules each comprise a pyrimidine nucleobase, and the first functional group is attached directly or indirectly to the C5 position of the nucleobase of each of the plurality of modified nucleotide molecules.

In some instances, the first functional group can be attached directly or indirectly to the sugar of each of the plurality of modified nucleotide molecules, optionally wherein the first functional group is attached directly or indirectly to 3′ position or 5′ position of the sugar moiety.

Methods for preparing azide-modified nucleosides are described in, for example, Müggenburg et al. (2022), “Azide-Modified Nucleosides as Versatile Tools for Bioorthogonal Labeling and Functionalization”, Chem. Rec. 22, c202100322, p. 1-13, which is incorporated herein by reference in its entirety. For example, 5′-azidoadenosine can be synthesized from commercially-available 5′-chloro adenosine in a single reaction step. Alternatively, introduction of the azido functionality can be performed after activation of the 5′-hydroxyl group with methanesulfonyl chloride. Furthermore, a one pot synthesis 5′-azide-modified nucleosides starting from 2′,3′-protected ribonucleosides using PPh3 and CBr4 as halogenating agents that has excellent yields has been described by Peterson et al. (2014), “A Tractable and Efficient One-Pot Synthesis of 5′-Azido-5′-deoxyribonucleosides”, Molecules 19:2434-2444, which is incorporated herein by reference in its entirety. This strategy is compatible with all four standard ribonucleosides. Many of these reactions are also compatible with the preparation of azide modified nucleotides as well.

In some instances, the second functional group can be attached directly or indirectly to the nucleobase of the modified 3′ reversibly terminated nucleotide. In some instances, for example, the nucleobase is a purine, and the second functional group is attached directly or indirectly to the C7 position of the nucleobase. In some instances, the nucleobase is a pyrimidine, and the second functional group is attached directly or indirectly to the C5 position of the nucleobase.

In some instances, the second functional group can be attached directly or indirectly to the sugar of the modified 3′ reversibly terminated nucleotide, optionally wherein the second functional group is attached directly or indirectly to 3′ position or 5′ position of the sugar.

In some instances, 3′ reversibly terminated nucleotide can be modified with an azido group at 3′ position, and serves as both the reversible terminator and the click chemistry functional group.

In some instances, for example, the first functional group and second functional group are selected from a list of click reactive functional group pairs comprising: azido/alkynyl groups; alkynyl/azido groups; azido/dibenzocyclooctynyl (DBCO) groups; dibenzocyclooctynyl (DBCO)/azido groups; azido/cyclooctynyl groups; cyclooctynyl/azido groups; tetrazine/dienophile groups; dienophile/tetrazine groups; thiol/alkynyl groups; alkynyl/thiol groups; cyano/1,2-amino thiol groups; 1,2-amino thiol/cyano groups; nitrone/cyclooctynyl groups; cyclooctynyl/nitrone groups; or any combination thereof.

In some instances, the first functional group comprises an azido group and the second functional group comprises an alkynyl group. In some instances, the first functional group comprises an alkynyl group and the second functional group comprises an azido group. In some instances, the first functional group comprises an azido group and the second functional group comprises a cyclooctynyl group. In some instances, the first functional group comprises a cyclooctynyl group and the second functional group comprises an azido group. In some instances, the first functional group comprises a tetrazine group and the second functional group comprises a dienophile group. In some instances, the first functional group comprises a dienophile group and the second functional group comprises a tetrazine group. In some instances, the first functional group comprises a thiol group and the second functional group comprises an alkynyl group. In some instances, the first functional group comprises an alkynyl group and the second functional group comprises a thiol group. In some instances, the first functional group comprises a cyano group and the second functional group comprises a 1,2-amino thiol group. In some instances, the first functional group comprises a 1,2-amino thiol group and the second functional group comprises a cyano group. In some instances, the first functional group comprises a nitrone group and the second functional group comprises a cyclooctynyl group. In some instances, the first functional group comprises a cyclooctynyl group and the second functional group comprises a nitrone group. In some instances, the first functional group comprises tetrazine and the second functional group comprises trans-cyclooctene. In some instances, the first functional group comprises trans-cyclooctene and the second functional group comprises tetrazine.

In some instances, the first functional group comprises an azido group and the second functional group comprises a dibenzocyclooctynyl (DBCO) group. In some instances, the azido group is attached directly or indirectly to a base in the first plurality of modified nucleotide molecules. In some instances, the azido group is attached directly or indirectly to a 3′ position of a sugar in the first plurality of modified nucleotide molecules. In some instances, the azido group is attached directly or indirectly to a 5′ position of a sugar in the first plurality of modified nucleotide molecules. In some instances, the dibenzocyclooctynyl (DBCO) group is attached directly or indirectly to a base in the modified 3′ reversibly terminated nucleotide. In some instances, the dibenzocyclooctynyl (DBCO) group is attached directly or indirectly to a 3′ position of a sugar in the modified 3′ reversibly terminated nucleotide. In some instances, the dibenzocyclooctynyl (DBCO) group is attached directly or indirectly to a 5′ position of a sugar in the modified 3′ reversibly terminated nucleotide. In some instances, the dibenzocyclooctynyl (DBCO) group is attached directly or indirectly to a reversible blocking group of the modified 3′ reversibly terminated nucleotide.

In some instances, the first functional group comprises a dibenzocyclooctynyl (DBCO) group and the second functional group comprises an azido group. In some instances, the dibenzocyclooctynyl (DBCO) group is attached directly or indirectly to a base in the first plurality of modified nucleotide molecules. In some instances, the dibenzocyclooctynyl (DBCO) group is attached directly or indirectly to a 3′ position of a sugar in the first plurality of modified nucleotide molecules. In some instances, the dibenzocyclooctynyl (DBCO) group is attached directly or indirectly to a 5′ position of a sugar in the first plurality of modified nucleotide molecules. In some instances, the azido group is attached directly or indirectly to a base in the modified 3′ reversibly terminated nucleotide. In some instances, the azido group is attached directly or indirectly to a 3′ position of a sugar in the modified 3′ reversibly terminated nucleotide. In some instances, the azido group is attached directly or indirectly to a 5′ position of a sugar in the modified 3′ reversibly terminated nucleotide. In some instances, the azido group is attached directly or indirectly to a reversible blocking group of the modified 3′ reversibly terminated nucleotide.

In some instances, the first modified nucleotide molecule and the modified 3′ reversibly terminated nucleotide are bioconjugated via the first and second click reactive functional groups using an enzyme-free click reaction. In some instances, the click chemistry reaction is biorthogonal. In some instances the click reactive functional groups described herein comprise at one μM concentration per click functional group, a click reaction with a k₂ rate (M⁶s⁻¹) and associated half-life of 10⁵ and 10 seconds, 10⁴ and 1.7 minutes, 10³ and 17 minutes, 10² and 2.8 hours, 10 and 1.2 days, 1 and 12 days, 10⁻¹ and four months, or 10⁻² and three years.

In some instances, the click chemistry reaction is a nucleophilic addition reaction. In some instances, the click chemistry reaction is a cyclopropane-tetrazine reaction. In some instances, the click chemistry reaction is a strain-promoted azide-alkyne cycloaddition (SPAAC) reaction. In some instances, the click chemistry reaction is an alkyne hydrothiolation reaction. In some instances, the click chemistry reaction is an alkene hydrothiolation reaction. In some instances, the click chemistry reaction is a strain-promoted alkyne-nitrone cycloaddition (SPANC) reaction. In some instances, the click chemistry reaction is an inverse electron-demand Diels-Alder (IED-DA) reaction. In some instances, the click chemistry reaction is a cyanobenzothiazole condensation reaction. In some instances, the click chemistry reaction is an aldehyde/ketone condensation reaction. In some instances, the click chemistry reaction is a Cu(I)-catalyzed azide-alkyne cycloaddition (CuAAC) reaction.

In some instances, the coupling reaction is performed under suitable conditions for the respective click chemistry reaction to take place. In some instances, the coupling reaction is a spontaneous reaction (e.g., without requiring light, heat, or a radical). In some instances, the coupling reaction can occur in water, aqueous buffer, or cell culture media. In some instances, the coupling reaction is performed at physiological pH (e.g., about 7.4). In some instances, the coupling reaction can occur in organic solvents. In some instances, the coupling reaction is performed in the presence of a catalyst, e.g., copper.

In some instances, after performing the coupling reaction and prior to detecting the presence of a first modified nucleotide molecule coupled to the modified 3′ reversibly terminated nucleotide, the method further comprises a first wash step to remove unbound polymerase and unbound modified nucleotide molecules. In some instances, the first wash conditions are configured to not disrupt the bound complex. In some instances, the first wash step may comprise, for example, use of the same buffer used for contacting the primed template nucleic acid with a polymerase and nucleotide molecules (but without the polymerase and nucleotide molecules). In some instances, the first wash buffer may not include KCl and/or may include little to no DMSO. In some instances, the first wash buffer is similar to those used for wash buffers as used in wash steps of a Western blot (e.g., a wash buffer added in a Western blot after binding a primary antibody but washing prior to incubation with a secondary antibody, such as PBST). PBST is a phosphate-buffered saline with a low-concentration of detergent, such as 0.05% to 0.1% Tween.

In some instances, the method further comprises a second wash step after performing the detecting step to disrupt the complex. In some instances, the second wash is performed under more stringent conditions than the first wash. For example, the second wash may include a temperature higher than room temperature (e.g., 30-40° C.), a higher salt concentration (e.g., a higher KCl salt concentrations (e.g., at least 50 mM KCl)), a solvent miscible in the wash buffer solution (e.g., dimethyl sulfoxide (DMSO)), a detergent (e.g., sodium dodecyl sulfate (SDS)), or a combination thereof.

### Linkers

In some instances, the first functional group of each of the first plurality of modified nucleotides is attached to the modified nucleotide using a first linker. In some instances, the first linker comprises a polymer chain. In some instances, the polymer chain comprises at least 2, at least 5, at least 8, at least 10, at least 12, at least 15, at least 20, at least 25, at least 50, at least 75, or at least 100 monomer units. In some instances, the polymer chain is polyethylene glycol, polypropylene glycol, polyvinyl acetate, polylactic acid, or polyglycolic acid. In some instances, the polymer chain is a linear or branched molecule. In some instances, the first linker comprises a polyethylene glycol (PEG) linker. In some instances, the PEG linker comprises at least 2, at least 5, at least 8, at least 10, at least 12, at least 15, at least 20, at least 25, at least 50, at least 75, or at least 100 ethylene glycol units. In some instances, the PEG linker comprises 2 to 24 ethylene glycol units. In some instances, the first linker comprises a cleavable linker. In some instances, the cleavable linker comprises a photocleavable linker, a Pd-cleavable linker, a phosphine-cleavable linker, or a disulfide linker.

In some instances, the second functional group is attached to the modified 3′ reversibly terminated nucleotide using a second linker. In some instances, the second linker comprises a polymer chain. In some instances, the polymer chain comprises at least 2, at least 5, at least 8, at least 10, at least 12, at least 15, at least 20, at least 25, at least 50, at least 75, or at least 100 monomer units. In some instances, the polymer chain is polyethylene glycol, polypropylene glycol, polyvinyl acetate, polylactic acid, or polyglycolic acid. In some instances, the polymer chain is a linear or branched molecule. In some instances, the second linker comprises a polyethylene glycol (PEG) linker. In some instances, the PEG linker comprises at least 2, at least 5, at least 8, at least 10, at least 12, at least 15, at least 20, at least 25, at least 50, at least 75, or at least 100 ethylene glycol units. In some instances, the PEG linker comprises 2 to 24 ethylene glycol units. In some instances, the second linker comprises a cleavable linker. In some instances, the cleavable linker comprises a photocleavable linker, a Pd-cleavable linker, a phosphine-cleavable linker, or a disulfide linker. In some embodiments. In some instances, the second linker and the first linker are the same.

In some instances, the first and/or second cleavable linker comprises a photocleavable linker. Any suitable photocleavable linker can be used (see, e.g., Sco et al. (2005), PNAS 102 (17): 5926-5931, incorporated by reference herein in its entirety). In some instances, the photocleavable linker comprises a nitrobenzyl group. For instance, a photocleavable nitrobenzyl linker can be cleaved using laser irradiation (355 nm, 10 seconds, 1.5 Wcm⁻²).

In some instances, the first and/or second cleavable linker comprises a Pd-cleavable linker. Any suitable Pd-cleavable linker can be used (see, e.g., Ju et al. (2006), PNAS 103 (52): 19635-19640, incorporated by reference herein in its entirety). In some instances, the Pd-cleavable linker comprises an allyl group. For instance, a Pd-cleavable allyl linker can be cleaved using incubation with a Na₂PdCl₄/P(PhSO₃Na)₃ mixture (30 seconds at 70° C.).

In some instances, the first and/or second cleavable linker comprises a phosphine-cleavable linker. Any suitable phosphine-cleavable linker can be used (see, e.g., Guo et al. (2008), PNAS 105 (27): 9145-9150, incorporated by reference herein in its entirety). In some instances, the phosphine-cleavable linker comprises an azide group. For instance, a phosphine-cleavable azide linker can be cleaved using incubation with a Tris (2-carboxyethyl) phosphine (TCEP) mixture (15 minutes at 65° C.).

In some instances, the first and/or second cleavable linker comprises a disulfide bond. For instance, the disulfide bond can be cleaved using incubation with a reducing agent, such as beta-mercaptoethanol, TCEP, or dithiothreitol (DTT).

### Polymerases

Examples of polymerases that may be used for performing the disclosed methods include, but are not limited to, DNA polymerases (e.g., Taq DNA polymerase), RNA polymerases, and/or reverse transcriptases.

In some aspects, the polymerase of the polymerase conjugate is a DNA polymerase. Examples of DNA polymerases include Taq polymerase, 9°N-7 DNA polymerase (or variants thereof, for example, D141A/E143A/A485L), phi29 (φ29) polymerase, Klenow fragment, Bacillus stearothermophilus DNA polymerase (BST), T4 DNA polymerase, T7 DNA polymerase, and DNA polymerase I. In some aspects, DNA polymerases that have been engineered or mutated to have desirable characteristics can be employed. In some aspects, the polymerase is phi29 DNA polymerase. In some aspects, the polymerase of the polymerase conjugate is a DNA polymerase and the template nucleic acid molecule includes DNA. In some aspects, the polymerase of the polymerase conjugate is a DNA polymerase and the nucleotide molecules include deoxyribonucleotides.

In some aspects, the DNA polymerase is Taq polymerase or a functional variant thereof. Taq polymerase is a heat stable polymerase from Thermus aquaticus. An example Taq polymerase sequence is:

(SEQ ID NO: 1)
GMLPLFEPKGRVLLVDGHHLAYRTFHALKGLTTSRGEPVQAVYGFAKSL
LKALKEDGDAVIVVFDAKAPSFRHEAYGGYKAGRAPTPEDFPRQLALIK
ELVDLLGLARLEVPGYEADDVLASLAKKAEKEGYEVRILTADKDLYQLL
SDRIHVLHPEGYLITPAWLWEKYGLRPDQWADYRALTGDESDNLPGVKG
IGEKTARKLLEEWGSLEALLKNLDRLKPAIREKILAHMDDLKLSWDLAK
VRTDLPLEVDFAKRREPDRERLXAFLERLEFGSLLHEFGLLESPKXLXE
APWPPPERAFVP.

In some aspects, the DNA polymerase is phi29 DNA polymerase or a functional variant thereof. The DNA polymerase of phi29 (a phage of Bacillus subtilis) has high processivity and fidelity. An example phi29 DNA polymerase sequence is:

(SEQ ID NO: 2)
MKHMPRKMYSCDFETTTKVEDCRVWAYGYMNIEDHSEYKIGNSLDEFMA
WVLKVQADLYFHNLKFDGAFIINWLERNGFKWSADGLPNTYNTIISRMG
QWYMIDICLGYKGKRKIHTVIYDSLKKLPFPVKKIAKDFKLTVLKGDID
YHKERPVGYKITPEEYAYIKNDIQIIAEALLIQFKQGLDRMTAGSDSLK
GFKDIITTKKFKKVFPTLSLGLDKEVRYAYRGGFTWLNDRFKEKEIGEG
MVFDVNSLYPAQMYSRLLPYGEPIVFEGKYVWDEDYPLHIQHIRCEFEL
KEGYIPTIQIKRSRFYKGNEYLKSSGGEIADLWLSNVDLELMKEHYDLY
NVEYISGLKFKATTGLFKDFIDKWTYIKTTSEGAIKQLAKLMLNSLYGK
FASNPDVTGKVPYLKENGALGFRLGEEETKDPVYTPMGVFITAWARYTT
ITAAQACYDRIIYCDTDSIHLTGTEIPDVIKDIVDPKKLGYWAHESTFK
RAKYLRQKTYIQDIYMKEVDGKLVEGSPDDYTDIKFSVKCAGMTDKIKK
EVTFENFKVGFSRKMKPKPVQVPGGVVLVDDTFTIK.

In some aspects, the DNA polymerase is a 9°N-7 DNA polymerase or a functional variant thereof (e.g., D141A/E143A/A485L). 9°N-7 is a strain of Thermococcus sp. An example of a 9°N-7 DNA polymerase sequence is:

(SEQ ID NO: 3)
MILDTDYITENGKPVIRVFKKENGEFKIEYDRTFEPYFYALLKDDSAIE
DVKKVTAKRHGTVVKVKRAEKVQKKFLGRPIEVWKLYFNHPQDVPAIRD
RIRAHPAVVDIYEYDIPFAKRYLIDKGLIPMEGDEELTMLAFDIETLYH
EGEEFGTGPILMISYADGSEARVITWKKIDLPYVDVVSTEKEMIKRFLR
VVREKDPDVLITYNGDNFDFAYLKKRCEELGIKFTLGRDGSEPKIQRMG
DRFAVEVKGRIHFDLYPVIRRTINLPTYTLEAVYEAVFGKPKEKVYAEE
IAQAWESGEGLERVARYSMEDAKVTYELGREFFPMEAQLSRLIGQSLWD
VSRSSTGNLVEWFLLRKAYKRNELAPNKPDERELARRRGGYAGGYVKEP
ERGLWDNIVYLDFRSLYPSIIITHNVSPDTLNREGCKEYDVAPEVGHKF
CKDFPGFIPSLLGDLLEERQKIKRKMKATVDPLEKKLLDYRQRAIKILA
NSFYGYYGYAKARWYCKECAESVTAWGREYIEMVIRELEEKFGFKVLYA
DTDGLHATIPGADAETVKKKAKEFLKYINPKLPGLLELEYEGFYVRGFF
VTKKKYAVIDEEGKITTRGLEIVRRDWSEIAKETQARVLEAILKHGDVE
EAVRIVKEVTEKLSKYEVPPEKLVIHEQITRDLRDYKATGPHVAVAKRL
AARGVKIRPGTVISYIVLKGSGRIGDRAIPADEFDPTKHRYDAEYYIEN
QVLPAVERILKAFGYRKEDLRYQKTKQVGLGAWLKVKGKK.

In some aspects, the DNA polymerase is DNA polymerase I or a functional fragment thereof (e.g., a Klenow fragment). Klenow fragment is an exonuclease deficient fragment of DNA polymerase I. An example of DNA polymerase I sequence is:

(SEQ ID NO: 4)
MVQIPQNPLILVDGSSYLYRAYHAFPPLTNSAGEPTGAMYGVLNMLRSL
IMQYKPTHAAVVFDAKGKTFRDELFEHYKSHRPPMPDDLRAQIEPLHAM
VKAMGLPLLAVSGVEADDVIGTLAREAEKAGRPVLISTGDKDMAQLVTP
NITLINTMTNTILGPEEVVNKYGVPPELIIDFLALMGDSSDNIPGVPGV
GEKTAQALLQGLGGLDTLYAEPEKIAGLSFRGAKTMAAKLEQNKEVAYL
SYQLATIKTDVELELTCEQLEVQQPAAEELLGLFKKYEFKRWTADVEAG
KWLQAKGAKPAAKPQETSVADEAPEVTATVISYDNYVTILDEETLKAWI
AKLEKAPVFAFDTETDSLDNISANLVGLSFAIEPGVAAYIPVAHDYLDA
PDQISRERALELLKPLLEDEKALKVGQNLKYDRGILANYGIELRGIAFD
TMLESYILNSVAGRHDMDSLAERWLKHKTITFEEIAGKGKNQLTFNQIA
LEEAGRYAAEDADVTLQLHLKMWPDLQKHKGPLNVFENIEMPLVPVLSR
IERNGVKIDPKVLHNHSEELTLRLAELEKKAHEIAGEEFNLSSTKQLQT
ILFEKQGIKPLKKTPGGAPSTSEEVLEELALDYPLPKVILEYRGLAKLK
STYTDKLPLMINPKTGRVHTSYHQAVTATGRLSSTDPNLQNIPVRNEEG
RRIRQAFIAPEDYVIVSADYSQIELRIMAHLSRDKGLLTAFAEGKDIHR
ATAAEVFGLPLETVTSEQRRSAKAINFGLIYGMSAFGLARQLNIPRKEA
QKYMDLYFERYPGVLEYMERTRAQAKEQGYVETLDGRRLYLPDIKSSNG
ARRAAAERAAINAPMQGTAADIIKRAMIAVDAWLQAEQPRVRMIMQVHD
ELVFEVHKDDVDAVAKQIHQLMENCTRLDVPLLVEVGSGENWDQAH.
In some aspects, a Klenow fragment includes
positions 324-928 with respect to SEQ ID NO: 4.

In some aspects, the polymerase of the polymerase conjugate is a reverse transcriptase. Reverse transcriptases typically have RNA-dependent DNA polymerase activity and DNA-dependent DNA polymerase activity. Examples of reverse transcriptases include Moloney murine leukemia virus (MMLV) reverse transcriptase, HIV-1 reverse transcriptase, and avian myeloblastosis virus (AMV) reverse transcriptase. In some aspects, the reverse transcriptase lacks (e.g., is mutated to lack) ribonuclease activity. In some instances, ribonuclease activity may degrade template particularly during longer incubation times such as when reverse transcribing longer cDNAs. In some aspects, the polymerase of the polymerase conjugate is a reverse transcriptase and the template nucleic acid molecule is an RNA molecule. In some aspects, the polymerase of the polymerase conjugate is a reverse transcriptase and the nucleotide molecules include deoxyribonucleotide molecules.

In some aspects, the reverse transcriptase is an MMLV reverse transcriptase or a functional variant thereof. An example of an MMLV reverse transcriptase sequence is:

(SEQ ID NO: 5)
AFPLERPDWDYTTQAGRNHLVHYRQLLLAGLQNAGRSPTNLAKVKGITQ
GPNESPSAFLERLKEAYRRYTPYDPEDPGQETNVSMSFIWQSAPDIGRK
LGRLEDLKSKTLGDLVREAEKIFNKRETPEEREERIRRETEEKEERRRT
VDEQKEKERDRRRHREMSKLLATVVIGQEQDRQEGERKRPQLDKDQCAY
CKEKGHWAKDCPKKPRGPRGPRPQTSLLTLGDXGGQGQDPPPEPRITLK
VGGQPVTFLVDTGAQHSVLTQNPGPLSDKSAWVQGATGGKRYRWTTDRK
VHLATGKVTHSFLHVPDCPYPLLGRDLLTKLKAQIHFEGSGAQVVGPMG
QPLQVLTLNIEDEYRLHETSKEPDVSLGFTWLSDFPQAWAESGGMGLAV
RQAPLIIPLKATSTPVSIKQYPMSQEARLGIKPHIQRLLDQGILVPCQS
PWNTPLLPVKKPGTNDYRPVQDLREVNKRVEDIHPTVPNPYNLLSGLPP
SHQWYTVLDLKDAFFCLRLHPTSQPLFAFEWRDPEMGISGQLTWTRLPQ
GFKNSPTLFDEALHRDLADFR. Residues 431-560 of SEQ
ID NO: 5 provide reversetranscriptase activity.

In some aspects, the reverse transcriptase is an HIV-1 reverse transcriptase or a functional variant thereof. An example of an HIV-1 reverse transcriptase sequence is:

(SEQ ID NO: 6)
PISPIEPVPVKLKPGMDGPKVKQWPLTEEKIKALVEICTEMEKEGKISK
IGPENPYNTPVFAIKKKDSTRWRKLVDFRELNKRTQDFWEVQLGIPHPA
GLKKKRSVTVLDVGDAYFSVPLDKEFRKYTAFTIPSINNETPGIRYQYN
VLPQGWKGSPAIFQSSMTKILEPFRKQNPDIVIYQYMDDLYVGSDLEIG
QHRTKIEELRQHLLKWGFTTPDKKHQKEPPFLWMGYEHHPDKWTVQPIV
LPEKDSWTVNDIQK.

In some instances, the polymerase is selected from Taq polymerase, 9°N-7 DNA polymerase or a functional variant thereof (e.g., D141A/E143A/A485L), and a Klenow fragment of DNA polymerase I. In some instances, the polymerase is not labeled with a detectable label.

### Workflows for Performing Click Chemistry-Based Sequencing

The disclosed click chemistry-based sequencing methods may be applied to both in situ sequencing and flow cell sequencing applications, where the click chemistry-based sequencing reactions are substituted for the stepwise nucleotide incorporation reactions used to probe a template nucleic acid sequence in, e.g., a conventional sequencing-by-synthesis (SBS) method.

In the case of in situ sequencing, the disclosed methods may comprise performing all or a subset of the following steps (in addition to a cyclic series of click chemistry-based base-by-base sequencing reactions):
(a) preparing the biological sample (e.g., by fixing, sectioning, embedding, and/or clearing a cell or tissue sample, as described elsewhere herein).
(b) contacting target analytes (e.g., target nucleic acid analytes and/or protein analytes) within the prepared sample with target-specific probes, as described elsewhere herein. In some instances, the target-specific probes may comprise, e.g., target-specific linear and/or circularizable nucleic acid probes (e.g., padlock probes) designed to hybridize directly or indirectly to specific target nucleic acid analytes. In some instances, the target-specific linear and/or circularizable nucleic acid probes may optionally comprise primer binding sites and/or target-specific barcode (or identifier) sequences. In some instances, the target-specific probes may comprise, e.g., target-specific antibodies designed to bind to specific target protein analytes, where the antibodies are conjugated to nucleic acid sequences. In some instances, the conjugated nucleic acid sequences may optionally comprise primer binding sites and/or target-specific barcode (or identifier) sequences.
(c) optionally performing a reverse transcription reaction (e.g., if the probed target nucleic acid analytes comprise RNA molecules) to create cDNA copies of RNA target molecules.
(d) optionally amplifying the probed target analyte molecules and/or their associated target-specific barcode sequences (e.g., using rolling circle amplification (RCA) in the case that target-specific circularizable probes were used to probe target analyte molecules and/or associated barcode sequences).
(e) contacting the optionally amplified target nucleic acid analytes and/or associated target-specific barcode sequences with sequencing primers designed to hybridize directly or indirectly to the target nucleic acid analytes and/or their associated target-specific barcode sequences. In some instances, the sequencing primers may comprise modified, 3′ reversibly terminated nucleotides that comprise a second functional group (e.g., a click chemistry reactive group) at their 3′ termini, thereby blocking the incorporation of nucleotides into the sugar-phosphate backbone of the priming strand when contacting primed template nucleic acid molecules (e.g., target nucleic acid molecules and/or associated target-specific barcode sequences) with a polymerase and a modified nucleotide that comprises a first functional group (e.g., a click reaction group that is reactive with the second function group). In some instances, the sequencing primers may comprise free 3′-hydroxyl groups at their 3′ termini, and an initial primer extension reaction may be performed to incorporate 3′ reversibly terminated nucleotides that comprise the second functional group at 3′ termini of the bound primers (i.e., 3′ termini of the priming strands).

In the case of flow cell sequencing, the disclosed methods may comprise performing all or a subset of the following steps (in addition to a cyclic series of click chemistry-based base-by-base sequencing reactions):
(a) extraction and purification of nucleic acid molecules (e.g., endogenous nucleic acid sequences) from a biological sample, as described elsewhere herein.
(b) preparation of a sequencing library comprising template nucleic acid molecules (e.g., the endogenous nucleic acid sequences or fragments thereof) that have been end-repaired and ligated to adapter sequences, as described elsewhere herein.
(c) optionally performing nucleic acid amplification of all or a portion of the sequencing library, as described elsewhere herein.
(d) immobilizing the template nucleic acid molecules (e.g., denatured, single-stranded template nucleic acid molecules) from the sequencing library on an inner surface of a flow cell using capture probes (e.g., complementary adapter sequences) that have been tethered to the flow cell surface.
(e) performing clonal amplification of the immobilized template nucleic acid molecules to create clusters comprising, e.g., thousands or tens of thousands of copies of the template nucleic acid molecule immobilized at each of a plurality of locations on the flow cells surface.
(f) contacting the template nucleic acid molecules in each clonally-amplified cluster with sequencing primers designed to hybridize to, e.g., the adapter sequences ligated to the template nucleic acid molecules. In some instances, the sequencing primers may comprise modified, 3′ reversibly terminated nucleotides that comprise a second functional group (e.g., a click chemistry reactive group) at their 3′ termini, thereby blocking the incorporation of nucleotides into the sugar-phosphate backbone of the priming strand when contacting primed template nucleic acid molecules with a polymerase and a plurality of modified nucleotides that comprise a first functional group (e.g., a click reaction group that is reactive with the second function group). In some instances, the sequencing primers may comprise free 3′-hydroxyl groups at their 3′ termini, and an initial primer extension reaction may be performed to incorporate 3′ reversibly terminated nucleotides that comprise the second functional group at 3′ termini of the bound primers (i.e., 3′ termini of the priming strands).

In the case of either in situ sequencing or flow cell sequencing, the disclosed methods can comprise: performing a cyclic series of click chemistry-based base-by-base sequencing reactions, where each sequencing cycle comprises:
(a) contacting each priming strand bound to a template nucleic acid molecule with a polymerase and a modified nucleotide (e.g., at least one modified nucleotide or a plurality of modified nucleotides) comprising the first functional group to form a complex comprising the 3′ terminus of the priming strand, the template nucleic acid molecule, the polymerase, and a modified nucleotide comprising the first functional group that is complementary to a nucleotide in the template nucleic acid molecule, where the modified nucleotide comprising the first functional group of the complex is not incorporated into the priming strand (i.e., is not incorporated into the sugar-phosphate backbone of the priming strand) because of the presence of 3′ reversibly terminated nucleotide (in some instances the at least one modified nucleotide or plurality of modified nucleotides comprising the first functional group further comprise detectably-labeled (e.g., fluorescently-labeled), modified nucleotide molecules comprising the first functional group);
(b) performing a coupling reaction between the first functional group and the second functional group to covalently couple the modified nucleotide molecule included in the complex to the modified 3′ reversibly terminated nucleotide of the priming strand; and
(c) detecting the presence of the modified nucleotide molecule coupled to the modified 3′ reversibly terminated nucleotide of the priming strand to identify a complementary nucleotide in the template nucleic acid molecule. In some instances, detecting the presence of the modified nucleotide molecule may comprise detecting a signal (e.g., a fluorescence signal) associated with a detectably-labeled modified nucleotide molecule (e.g., a fluorescently-labeled modified nucleotide molecule). In some instances, detecting the presence of the modified nucleotide molecule may comprise detecting an absence of signal (e.g., the modified nucleotide that is complementary to the nucleotide in the template nucleic acid molecule may not comprise a fluorophore or other detectable label).

In some instances, the disclosed methods may further comprise processing optical signals (e.g., fluorescence signals) detected in images (e.g., fluorescence images) acquired during the cyclic series of base-by-base sequencing reactions to detect the presence or absence of complementary fluorescently-labeled modified nucleotides coupled to the modified 3′ reversibly terminated nucleotide of the priming strand in each sequencing cycle at the locations of each of a plurality of template nucleic acid molecules (i.e., the locations corresponding to each of a plurality of target analyte molecules and/or their associated target-specific barcode sequences), thereby enabling inference of the nucleotide sequence of the plurality of template nucleic acid molecules (e.g., the plurality of target analyte molecules and/or associated target-specific barcode sequences).

In some instances, the cyclic series of click chemistry-based base-by-base sequencing reactions may comprise performing at least 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, or more than 50 cycles of the click chemistry-based base-by-base sequencing reaction.

In some instances, each cycle of base-by-base sequencing may further comprise a first wash step following the contacting step to remove unbound polymerase and modified nucleotide molecules. In some instances, the first wash conditions are configured to not disrupt the bound complex. In some instances, the first wash step may comprise, for example, use of the same buffer used for contacting the primed template nucleic acid with a polymerase and nucleotide molecules (but without the polymerase and nucleotide molecules). In some instances, the first wash buffer may not include KCl and/or may include little to no DMSO. In some instances, the first wash buffer is similar to those used for wash buffers as used in wash steps of a Western blot (e.g., a wash buffer added in a Western blot after binding a primary antibody but washing prior to incubation with a secondary antibody, such as PBST). PBST is a phosphate-buffered saline with a low-concentration of detergent, such as 0.05% to 0.1% Tween.

In some instances, each cycle of base-by-base sequencing may further comprise a second wash step following the detection step in order to disrupt the complex and remove the displaced polymerase. In some instances, the second wash is performed under more stringent conditions than the first wash. For example, the second wash may include a temperature higher than room temperature (e.g., 30-40° C.), a higher salt concentration (e.g., a higher KCl salt concentrations (e.g., at least 50 mM KCl)), a solvent miscible in the wash buffer solution (e.g., dimethyl sulfoxide (DMSO)), a detergent (e.g., sodium dodecyl sulfate (SDS)), or a combination thereof.

In some instances, each cycle of base-by-base sequencing may further comprise cleaving the covalently coupled modified nucleotide molecules from the modified 3′ reversibly terminated nucleotides of the priming strand, deprotecting the modified 3′ reversibly terminated nucleotide at the 3′ termini of the priming strand, and performing a primer extension reaction to incorporate a cognate modified 3′ reversibly terminated nucleotide that comprises the second functional group, thereby generating an extended priming strand.

In some instances the detection step may comprise the use of an optical imaging technique (e.g., a fluorescence imaging technique) and real time or post-processing measurement of optical signals (e.g., fluorescence signals or the absence thereof) associated with the presence of a specific modified nucleotide molecule covalently coupled to the modified 3′ reversibly terminated nucleotide of the priming strand at a plurality of locations corresponding to a plurality of target analytes distributed throughout the biological sample or tethered to specific locations on a substrate surface (e.g., a flow cell surface).

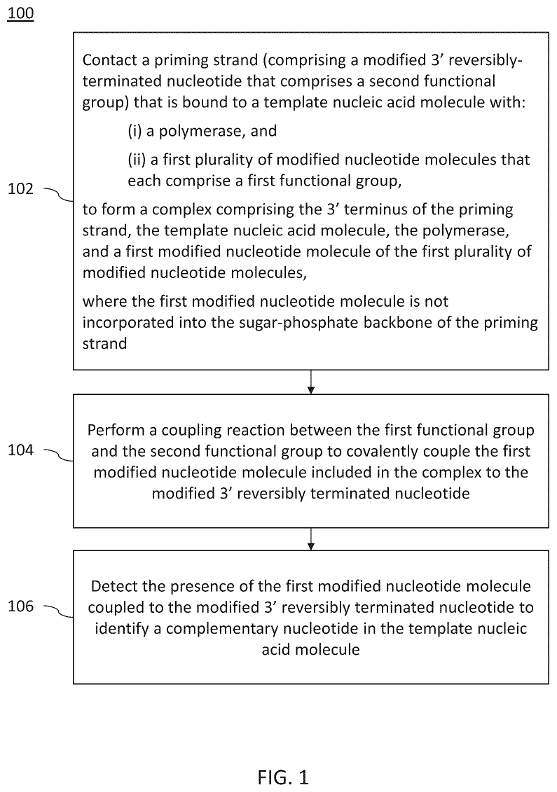

FIG. 1 provides a non-limiting example of a flowchart for a process 100 for sequencing a template nucleic acid molecule in accordance with one implementation of the methods described herein. The sequencing steps depicted in FIG. 1 may be performed as part of an in situ sequencing method or as part of a flow cell sequencing method. In process 100, some steps may optionally be combined, the order of some steps may optionally be changed, and some steps may optionally be omitted. In some instances, additional steps may be performed in combination with the steps shown in process 100. Accordingly, the steps illustrated (and described in greater detail below) for process 100 are exemplary by nature, and as such, should not be viewed as limiting.

At step 102 in FIG. 1, a priming strand bound to the template nucleic acid molecule is contacted with: (i) a polymerase and (ii) a first plurality of modified nucleotide molecules that each comprise a first functional group, to form a complex comprising a 3′ terminus of the priming strand, the template nucleic acid molecule, the polymerase, and a first modified nucleotide molecule of the first plurality of modified nucleotide molecules, where the priming strand comprises a modified 3′ reversibly terminated nucleotide that comprises a second functional group, and wherein the first modified nucleotide molecule is not incorporated into a sugar-phosphate backbone of the priming strand.

In some instances, the template nucleic acid molecule may comprise an endogenous nucleic acid molecule (e.g., a DNA molecule, an RNA molecule, or an mRNA molecule) that has been reverse transcribed, amplified, and/or extracted from a biological sample (e.g., a cell sample or tissue sample).

In some instances, the template nucleic acid molecule may comprise a barcode sequence (e.g., a nucleic acid barcode sequence) associated with a target analyte of interest (e.g., using the barcoding methods described elsewhere herein) that has been reverse transcribed, amplified, and/or extracted from a biological sample (e.g., a cell sample or tissue sample).

In some instances, the method may further comprise hybridizing a circularizable probe to a target analyte (or to a labeling agent bound to the target analyte), ligating the circularizable probe to form a circularized probe, and performing rolling circle amplification of the circularized probe to generate the template nucleic acid molecule. In some instances, for example, the circularizable probe may be a padlock probe sequence.

In some instances, the template nucleic acid molecule to be sequenced may be attached to a solid support, e.g., a sequencing flow cell.

In some instances, the template nucleic acid molecule is sequenced in situ in a cell sample or tissue sample. In some instances, the cell sample may comprise a layer of cells deposited on a surface.

In some instances, the method may further comprise providing: i) one or more reagents comprising the polymerase and the first plurality of modified nucleotide molecules (each comprising the first functional group), and ii) the priming strand bound to the template nucleic acid molecule, where the priming strand comprises a modified 3′reversibly terminated nucleotide (at the 3′ terminus of the priming strand) that comprises the second functional group.

In some instances, the polymerase may comprise, e.g., Taq polymerase, Therminator™ DNA polymerase, a Klenow fragment of DNA polymerase I, or any combination thereof. In some instances, the polymerase is not labeled with a detectable label.

In some instances, the (non-covalently bound) complex consisting of the 3′ terminus of the priming strand, the template nucleic acid molecule, a polymerase, and a modified nucleotide molecule may comprise a transient complex. In some instances, the transient complex may persist for at least 5 sec, 10 sec, 20 sec, 30 sec, 40 sec, 50 sec, 1 min, 2 min, 3 min, 4 min, 5 min, or 10 min after removal of polymerase-modified nucleotide mixture used to contact the primed template nucleic acid molecule and form the complex. The “persistence time” of the complex, as used herein, refers to the average length of time that the complex remains stable without significant dissociation of any of the components of the bound complex.

At step 104 in FIG. 1, a coupling reaction between the first functional group and the second functional group is performed to covalently couple the first modified nucleotide molecule included in the complex to the modified 3′ reversibly terminated nucleotide.

In some instances, the coupling reaction may comprise a click chemistry reaction (as described in more detail elsewhere herein). For example, the first functional group and second functional group may be selected from a list of click reactive functional group pairs including, but not limited to, azido/alkynyl groups, alkynyl/azido groups, azido/dibenzocyclooctynyl (DBCO) groups, dibenzocyclooctynyl (DBCO)/azido groups, azido/cyclooctynyl groups, cyclooctynyl/azido groups, tetrazine/dienophile groups, dienophile/tetrazine groups, thiol/alkynyl groups, alkynyl/thiol groups, cyano/1,2-amino thiol groups, 1,2-amino thiol/cyano groups, nitrone/cyclooctynyl groups, cyclooctynyl/nitrone groups, or any combination thereof. In some instances, the first plurality of modified nucleotides contacted with the primed template nucleic acid molecule (e.g., the template nucleic acid molecule with a bound priming strand), or the plurality of modified nucleotides contacted with the primed template nucleic acid in any cycle of a multicycle sequencing process, may comprise more than one click reactive functional group pairs, e.g., 2, 3, 4, or more than 4 click reactive functional group pairs.

In some instances, the first functional group of each of the first plurality of modified nucleotides may be attached to the modified nucleotide using a first linker, e.g., a polyethylene glycol (PEG) linker where the polyethylene glycol (PEG) linker comprises from 2 to 24 ethylene glycol units. In some instances, the first linker may comprise a cleavable linker, e.g., a photocleavable linker, a Pd-cleavable linker, a phosphine-cleavable linker, or a disulfide linker.

In some instances, the second functional group may be attached to the modified 3′ reversibly terminated nucleotide using a second linker, e.g., a cleavable linker such as a photocleavable linker, a Pd-cleavable linker, a phosphine-cleavable linker, or a disulfide linker. In some instances, the second linker and the first linker may be the same.

In some instances, for example, the first functional group may comprise an azido group and the second functional group may comprise a dibenzocyclooctynyl (DBCO) group. In some instances, the azido group may be attached directly or indirectly to a base (e.g., the nucleobase) in the first plurality of modified nucleotide molecules. In some instances, the azido group may be attached directly or indirectly to a 3′ position of a sugar in the first plurality of modified nucleotide molecules. In some instances, the azido group may be attached directly or indirectly to a 5′ position of a sugar in the first plurality of modified nucleotide molecules. In some instances, the dibenzocyclooctynyl (DBCO) group may be attached directly or indirectly to a base in the modified 3′ reversibly terminated nucleotide. In some instances, the dibenzocyclooctynyl (DBCO) group may be attached directly or indirectly to a 3′ position of a sugar in the modified 3′ reversibly terminated nucleotide. In some instances, the dibenzocyclooctynyl (DBCO) group may be attached directly or indirectly to a 5′ position of a sugar in the modified 3′ reversibly terminated nucleotide. In some instances, the dibenzocyclooctynyl (DBCO) group may be attached directly or indirectly to a reversible blocking group of the modified 3′ reversibly terminated nucleotide.

In other instances, the first functional group may comprise a dibenzocyclooctynyl (DBCO) group and the second functional group may comprise an azido group. In some instances, the dibenzocyclooctynyl (DBCO) group may be attached directly or indirectly to a base (e.g., the nucleobase) in the first plurality of modified nucleotide molecules. In some instances, the dibenzocyclooctynyl (DBCO) group may be attached directly or indirectly to a 3′ position of a sugar in the first plurality of modified nucleotide molecules. In some instances, the dibenzocyclooctynyl (DBCO) group may be attached directly or indirectly to a 5′ position of a sugar in the first plurality of modified nucleotide molecules. In some instances, the azido group may be attached directly or indirectly to a base in the modified 3′ reversibly terminated nucleotide. In some instances, the azido group may be attached directly or indirectly to a 3′ position of a sugar in the modified 3′ reversibly terminated nucleotide. In some instances, the azido group may be attached directly or indirectly to a 5′ position of a sugar in the modified 3′ reversibly terminated nucleotide. In some instances, the azido group may be attached directly or indirectly to a reversible blocking group of the modified 3′ reversibly terminated nucleotide.

The options for coupling an azido group and a dibenzocyclooctynyl (DBCO) group to the modified nucleotides and 3′ reversibly terminated nucleotide listed in the preceding paragraphs are equally applicable to any other click reactive functional group pairs including, but not limited to, azido/alkynyl groups, alkynyl/azido groups, azido/dibenzocyclooctynyl (DBCO) groups, dibenzocyclooctynyl (DBCO)/azido groups, azido/cyclooctynyl groups, cyclooctynyl/azido groups, tetrazine/dienophile groups, dienophile/tetrazine groups, thiol/alkynyl groups, alkynyl/thiol groups, cyano/1,2-amino thiol groups, 1,2-amino thiol/cyano groups, nitrone/cyclooctynyl groups, cyclooctynyl/nitrone groups, or any combination thereof.

At step 106 in FIG. 1, the presence of the first modified nucleotide molecule coupled to the modified 3′ reversibly terminated nucleotide is detected to identify a complementary nucleotide in the template nucleic acid molecule.

In some instances, detecting a presence of a modified nucleotide molecule in the complex may comprise detecting a signal associated with a detectable label coupled to the modified nucleotide molecule. In some instances, detecting a presence of a modified nucleotide molecule in the complex may comprise detecting an absence of a signal, where the absence of signal is associated with a modified nucleotide molecule that is not coupled to a detectable label, e.g., a fluorophore. In some instances, the detection step may be performed, e.g., using a fluorescence imaging technique as described elsewhere herein.

In some instances, the first plurality of modified nucleotide molecules (or one or more additional pluralities of modified nucleotide molecules) may comprise a set of four different modified nucleotide molecules, where each different modified nucleotide molecule (e.g., each modified nucleotide molecule comprising a different nucleobase) is coupled to a different fluorophore.

In some instances, the first plurality of modified nucleotide molecules (or one or more additional pluralities of modified nucleotide molecules) may comprise a set of four different modified nucleotide molecules, where three of the four different modified nucleotide molecules are coupled to different fluorophores and one of the four different modified nucleotide molecules is not conjugated to a fluorophore.

In some instances, the first plurality of modified nucleotide molecules (or one or more additional pluralities of modified nucleotide molecules) may comprise a set of four different modified nucleotide molecules, where two of the four different modified nucleotides are coupled to different fluorophores, one of the four different modified nucleotide molecules is coupled to the two different fluorophores, and one of the four different modified nucleotide molecules is not conjugated to a fluorophore.

In some instances, the first plurality of modified nucleotides (or one or more additional pluralities of modified nucleotide molecules) may be selected from modified A, T, U, C, and G. In some instances, the first plurality of modified nucleotides (or one or more additional pluralities of modified nucleotide molecules) may be selected from modified A, T, C, and G.

In some instances, the method (or process) depicted in FIG. 1 may further comprise: d) performing a cleavage reaction to remove the covalently coupled modified nucleotide molecule from the modified 3′ reversibly terminated nucleotide (as described elsewhere herein); e) performing a deprotection reaction to deprotect the modified 3′ reversibly terminated nucleotide (e.g., using Tris(2-carboxyethyl) phosphine in aqueous solution to remove 3′-O-azidomethyl groups from 3′-O-azidomethyl reversibly terminated nucleotides); and f) performing an extension reaction to incorporate a modified 3′ reversibly terminated nucleotide that comprises the second functional group and that is complementary to the identified nucleotide in the template nucleic acid molecule into an extended priming strand. In some instances, the cleavage reaction and the deprotection reaction are the same reaction.

In some instances, the second functional group may be attached indirectly to 3′ reversibly terminated molecule via a cleavable linker, and performing the cleavage reaction may comprise contacting the complex with a reagent capable of cleaving the cleavable linker. For example, in some instances, the cleavable linker can comprise a disulfide linker, and the cleavage reagent can comprise a disulfide reducing agent, optionally wherein the disulfide reducing agent comprises Dithiothreitol (DTT). In some instances, the cleavable linker may comprise a photocleavable linker (e.g., cleavable using UV light), a peroxide-cleavable arylboronic acid linker, a palladium (Pd)-cleavable linker (e.g., a bi-functionalized propargyl carbamate linker cleavable using a palladium complex), a phosphine-cleavable linker (e.g., a phosphine-cleavable linker comprising a disulfide moiety that can be cleaved using water-soluble phosphines or phosphine (see, e.g., U.S. Pat. No. 10,487,102, which is incorporated herein by reference in its entirety)), etc.

In some instances, the method (or process) depicted in FIG. 1 may further comprise performing a first wash step to remove unbound polymerase and unbound modified nucleotide molecules prior to performing the detecting step, as described elsewhere herein. In some instances, the method may further comprise performing a second wash step after performing the detecting step to disrupt the complex, as described elsewhere herein.

In some instances, the method (or process) depicted in FIG. 1 may further comprise repeating steps (a)-(f) for at least one additional cycle using at least one additional plurality of modified nucleotide molecules that comprise the first functional group to detect a presence of a modified nucleotide molecule coupled to the modified 3′ reversibly terminated nucleotide and thereby identify at least one additional complementary nucleotide in the template nucleic acid molecule. In some instances, the at least one additional cycle comprises at least 2, 5, 10, 20, 30, 40, or 50 additional cycles.

In some instances, the method (or process) depicted in FIG. 1 may further comprise: prior to performing a first contacting step in (a), hybridizing a primer that does not comprise a modified 3′ reversibly terminated nucleotide at its 3′ end to a primer binding site in the template nucleic acid molecule; and performing an extension reaction to incorporate a modified 3′ reversibly terminated nucleotide that comprises the second functional group and that is complementary to a nucleotide in the template nucleic acid molecule into an extended primer strand to generate the priming strand.

In some instances, the first plurality of modified nucleotide molecules and at least one additional plurality of modified nucleotide molecules may comprise a same set of modified nucleotide molecules (e.g., modified nucleotides comprising the same set of nucleobases). In some instances, the first plurality of modified nucleotide molecules and at least one additional plurality of modified nucleotide molecules may comprise different sets of modified nucleotide molecules (e.g., modified nucleotides comprising different sets of nucleobases).

In some instances, the first plurality of modified nucleotide molecules and at least one additional plurality of modified nucleotide molecules may each comprise modified nucleotide molecules that do not include a 3′ reversible terminator moiety.

In some instance, the first plurality of modified nucleotide molecules and at least one additional plurality of modified nucleotide molecules may each comprise one or more modified nucleotide molecules that are coupled to a detectable label, e.g., a fluorophore. In some instances, modified nucleotide molecules that are coupled to a detectable label and that comprise a different nucleobase are coupled to a different detectable label, e.g., a different fluorophore.

In some instances, the first plurality of modified nucleotide molecules and at least one additional plurality of modified nucleotide molecules may each comprise at least one modified nucleotide molecule that is not labeled with a detectable label.

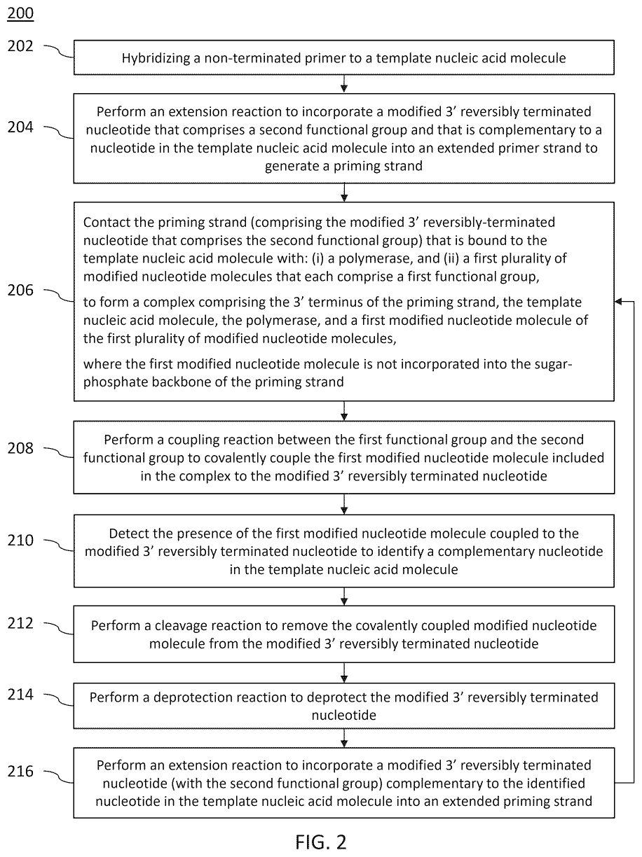

FIG. 2 provides a non-limiting example of a flowchart for a process 200 for sequencing a template nucleic acid molecule in accordance with one implementation of the methods described herein. The sequencing steps depicted in FIG. 2 may be performed as part of an in situ sequencing method or as part of a flow cell sequencing method. In process 200, some steps may optionally be combined, the order of some steps may optionally be changed, and some steps may optionally be omitted. In some instances, additional steps may be performed in combination with the steps shown in process 200. Accordingly, the steps illustrated (and described in greater detail below) for process 200 are exemplary by nature, and as such, should not be viewed as limiting.

At step 202 in FIG. 2, a non-terminated primer may be hybridized to a template nucleic acid molecule. In some instances, the template nucleic acid molecule may comprise an endogenous nucleic acid molecule (e.g., a DNA molecule, an RNA molecule, or an mRNA molecule) that has been reverse transcribed, amplified, and/or extracted from a biological sample (e.g., a cell sample or tissue sample).

In some instances, the template nucleic acid molecule may comprise a barcode sequence (e.g., a nucleic acid barcode sequence) associated with a target analyte of interest (e.g., using the barcoding methods described elsewhere herein) that has been reverse transcribed, amplified, and/or extracted from a biological sample (e.g., a cell sample or tissue sample).

In some instances, the method may further comprise hybridizing a circularizable probe to a target analyte (or to a labeling agent bound to the target analyte), ligating the circularizable probe to form a circularized probe, and performing rolling circle amplification of the circularized probe to generate the template nucleic acid molecule. In some instances, for example, the circularizable probe may be a padlock probe sequence. A template for rolling circle amplification can also be provided as a circularizable probe set (e.g., two or more nucleic acid molecules that can be ligated together to form a circular nucleic acid molecule, optionally wherein the two or more nucleic acid molecules are probes that can be ligated together upon hybridization to a target analyte or to a labeling agent bound to the target analyte). In some instances, the two or more nucleic acid molecules are ligated together using a first ligation templated by a target analyte or a labeling agent bound to the target analyte, and a second ligation templated by a splint oligonucleotide. In some instances, the method may comprise hybridizing a circularizable probe set to a target analyte (or to a labeling agent bound to the target analyte), ligating the circularizable probe set to form a circularized probe, and performing rolling circle amplification of the circularized probe to generate the template nucleic acid molecule. The ligation can be performed with or without gap-filling (e.g., as described elsewhere herein).

In some instances, the template nucleic acid molecule to be sequenced is a rolling circle amplification product in a biological sample or matrix. In some instances, the template nucleic acid molecule comprises multiple copies of a sequence of interest, and the methods described herein are applied to determine the sequence of interest. In some cases, the sequence of interest is 5′ and adjacent to a sequence capable of hybridizing to a priming strand (e.g., a sequencing primer). In some cases, the sequence of interest is a barcode sequence or complement thereof. In some cases, the sequence of interest is a sequence of a target nucleic acid that binds to a circularizable probe or probe set, wherein the circularizable probe or probe set is used as a template to generate the rolling circle amplification product. The circularizable probe or probe set may comprise the complement of the target nucleic acid sequence, or the complement of the target nucleic acid sequence may be incorporated into the circularizable probe or probe set by a gap-fill extension or ligation reaction. The rolling circle amplification product produced using the circularized probe or probe set as a template can thus comprise multiple copies of the original target nucleic acid sequence.

In some instances, the template nucleic acid molecule to be sequenced may be attached to a solid support, e.g., a sequencing flow cell. In some instances, the template nucleic acid molecule is sequenced in situ in a cell sample or tissue sample. In some instances, the cell sample may comprise a layer of cells deposited on a surface.

At step 204 in FIG. 2, an extension reaction is performed to incorporate a modified 3′ reversibly terminated nucleotide (that comprises a second functional group, and that is complementary to a nucleotide in the template nucleic acid molecule) into an extended primer strand to generate a priming strand.

In some instances, the modified 3′ reversibly terminated nucleotide may be, for example, a modified 3′-O-blocked reversibly terminated nucleotide. In some instances, the modified 3′-O-blocked reversibly terminated nucleotide may be, e.g., a modified 3′-O-azidomethyl deoxynucleotide triphosphate (3′-O-azidomethyl dNTP), a modified 3′-O-allyl deoxynucleotide triphosphate (3′-O-allyl-dNTP), or a modified 3′-O-amino deoxynucleotide triphosphate (3′-O—NH2 dNTP). In some instances, the modified 3′ reversibly terminated nucleotide may be a modified 3′-unblocked reversibly terminated nucleotide.

At step 206 in FIG. 2, the priming strand (comprising the modified 3′ reversibly terminated nucleotide that comprises the second functional group) that is bound to the template nucleic acid molecule is contacted with: (i) a polymerase, and (ii) a first plurality of modified nucleotide molecules (that each comprise a first functional group) to form a complex comprising the 3′ terminus of the priming strand, the template nucleic acid molecule, the polymerase, and a first modified nucleotide molecule of the first plurality of modified nucleotide molecules, where the first modified nucleotide molecule is not incorporated into the sugar-phosphate backbone of the priming strand.

In some instances, the method may further comprise providing: i) one or more reagents comprising the polymerase and the first plurality of modified nucleotide molecules (each comprising the first functional group), and ii) the priming strand bound to the template nucleic acid molecule, where the priming strand comprises a modified 3′reversibly terminated nucleotide (at the 3′ terminus of the priming strand) that comprises the second functional group.

In some instances, the polymerase may comprise, e.g., Taq polymerase, Therminator™ DNA polymerase, a Klenow fragment of DNA polymerase I, or any combination thereof. In some instances, the polymerase is not labeled with a detectable label.

In some instances, the (non-covalently bound) complex consisting of the 3′ terminus of the priming strand, the template nucleic acid molecule, a polymerase, and a modified nucleotide molecule may comprise a transient complex. In some instances, the transient complex may persist for at least 5 sec, 10 sec, 20 sec, 30 sec, 40 sec, 50 sec, 1 min, 2 min, 3 min, 4 min, 5 min, or 10 min after removal of polymerase-modified nucleotide mixture used to contact the primed template nucleic acid molecule and form the complex. The “persistence time” of the complex, as used herein, refers to the average length of time that the complex remains stable without significant dissociation of any of the components of the bound complex.

At step 208 in FIG. 2, a coupling reaction between the first functional group and the second functional group is performed to covalently couple the first modified nucleotide molecule included in the complex to the modified 3′ reversibly terminated nucleotide.

In some instances, as noted in reference to FIG. 1, the coupling reaction may comprise a click chemistry reaction (as described in more detail elsewhere herein). For example, the first functional group and second functional group may be selected from a list of click reactive functional group pairs including, but not limited to, azido/alkynyl groups, alkynyl/azido groups, azido/dibenzocyclooctynyl (DBCO) groups, dibenzocyclooctynyl (DBCO)/azido groups, azido/cyclooctynyl groups, cyclooctynyl/azido groups, tetrazine/dienophile groups, dienophile/tetrazine groups, thiol/alkynyl groups, alkynyl/thiol groups, cyano/1,2-amino thiol groups, 1,2-amino thiol/cyano groups, nitrone/cyclooctynyl groups, cyclooctynyl/nitrone groups, or any combination thereof. In some instances, the first plurality of modified nucleotides contacted with the primed template nucleic acid molecule (e.g., the template nucleic acid molecule with a bound priming strand), or the plurality of modified nucleotides contacted with the primed template nucleic acid in any cycle of a multicycle sequencing process, may comprise more than one click reactive functional group pairs, e.g., 2, 3, 4, or more than 4 click reactive functional group pairs.

In some instances, the first functional group of each of the first plurality of modified nucleotides may be attached to the modified nucleotide using a first linker, e.g., a polyethylene glycol (PEG) linker where the polyethylene glycol (PEG) linker comprises from 2 to 24 ethylene glycol units. In some instances, the first linker may comprise a cleavable linker, e.g., a photocleavable linker, a Pd-cleavable linker, a phosphine-cleavable linker, or a disulfide linker.

In some instances, the second functional group may be attached to the modified 3′ reversibly terminated nucleotide using a second linker, e.g., a cleavable linker such as a photocleavable linker, a Pd-cleavable linker, a phosphine-cleavable linker, or a disulfide linker. In some instances, the second linker and the first linker may be the same.

In some instances, for example, the first functional group may comprise an azido group and the second functional group may comprise a dibenzocyclooctynyl (DBCO) group. In some instances, the azido group may be attached directly or indirectly to a base (e.g., the nucleobase) in the first plurality of modified nucleotide molecules. In some instances, the azido group may be attached directly or indirectly to a 3′ position of a sugar in the first plurality of modified nucleotide molecules. In some instances, the azido group may be attached directly or indirectly to a 5′ position of a sugar in the first plurality of modified nucleotide molecules. In some instances, the dibenzocyclooctynyl (DBCO) group may be attached directly or indirectly to a base in the modified 3′ reversibly terminated nucleotide. In some instances, the dibenzocyclooctynyl (DBCO) group may be attached directly or indirectly to a 3′ position of a sugar in the modified 3′ reversibly terminated nucleotide. In some instances, the dibenzocyclooctynyl (DBCO) group may be attached directly or indirectly to a 5′ position of a sugar in the modified 3′ reversibly terminated nucleotide. In some instances, the dibenzocyclooctynyl (DBCO) group may be attached directly or indirectly to a reversible blocking group of the modified 3′ reversibly terminated nucleotide.

In other instances, the first functional group may comprise a dibenzocyclooctynyl (DBCO) group and the second functional group may comprise an azido group. In some instances, the dibenzocyclooctynyl (DBCO) group may be attached directly or indirectly to a base (e.g., the nucleobase) in the first plurality of modified nucleotide molecules. In some instances, the dibenzocyclooctynyl (DBCO) group may be attached directly or indirectly to a 3′ position of a sugar in the first plurality of modified nucleotide molecules. In some instances, the dibenzocyclooctynyl (DBCO) group may be attached directly or indirectly to a 5′ position of a sugar in the first plurality of modified nucleotide molecules. In some instances, the azido group may be attached directly or indirectly to a base in the modified 3′ reversibly terminated nucleotide. In some instances, the azido group may be attached directly or indirectly to a 3′ position of a sugar in the modified 3′ reversibly terminated nucleotide. In some instances, the azido group may be attached directly or indirectly to a 5′ position of a sugar in the modified 3′ reversibly terminated nucleotide. In some instances, the azido group may be attached directly or indirectly to a reversible blocking group of the modified 3′ reversibly terminated nucleotide.

The options for coupling an azido group and a dibenzocyclooctynyl (DBCO) group to the modified nucleotides and 3′ reversibly terminated nucleotide listed in the preceding paragraphs are equally applicable to any other click reactive functional group pairs including, but not limited to, azido/alkynyl groups, alkynyl/azido groups, azido/dibenzocyclooctynyl (DBCO) groups, dibenzocyclooctynyl (DBCO)/azido groups, azido/cyclooctynyl groups, cyclooctynyl/azido groups, tetrazine/dienophile groups, dienophile/tetrazine groups, thiol/alkynyl groups, alkynyl/thiol groups, cyano/1,2-amino thiol groups, 1,2-amino thiol/cyano groups, nitrone/cyclooctynyl groups, cyclooctynyl/nitrone groups, or any combination thereof.

At step 210 in FIG. 2, the presence of the first modified nucleotide molecule coupled to the modified 3′ reversibly terminated nucleotide is detected to identify a complementary nucleotide in the template nucleic acid molecule.

In some instances, detecting a presence of a modified nucleotide molecule in the complex may comprise detecting a signal associated with a detectable label coupled to the modified nucleotide molecule. In some instances, detecting a presence of a modified nucleotide molecule in the complex may comprise detecting an absence of a signal, where the absence of signal is associated with a modified nucleotide molecule that is not coupled to a detectable label, e.g., a fluorophore. In some instances, the detection step may be performed, e.g., using a fluorescence imaging technique as described elsewhere herein.

At step 212 in FIG. 2, a cleavage reaction is performed to remove the covalently coupled modified nucleotide molecule from the modified 3′ reversibly terminated nucleotide, as described elsewhere herein.

At step 214 in FIG. 2, a deprotection reaction to deprotect the modified 3′ reversibly terminated nucleotide (e.g., using Tris(2-carboxyethyl) phosphine in aqueous solution to remove 3′-O-azidomethyl groups from 3′-O-azidomethyl reversibly terminated nucleotides).

In some instances, the cleavage reaction and the deprotection reaction may be performed as a single reaction.

At step 216 in FIG. 2, an extension reaction is performed to incorporate a modified 3′ reversibly terminated nucleotide (with the second functional group) complementary to the identified nucleotide in the template nucleic acid molecule into an extended priming strand.

In some instances, the method (or process) depicted in FIG. 2 may further comprise performing a first wash step to remove unbound polymerase and unbound modified nucleotide molecules prior to performing the detecting step. In some instances, the method may further comprise performing a second wash step after performing the detecting step to disrupt the complex.

As indicated in FIG. 2, steps 206 to 216 may then be repeated for at least one additional cycle using at least one additional plurality of modified nucleotide molecules to detect a modified nucleotide molecule coupled to the modified 3′ reversibly terminated nucleotide and identify at least one additional complementary nucleotide in the template nucleic acid molecule. In some instances, the at least one additional cycle may comprise, e.g., at least 2, 5, 10, 20, 30, 40, 50, or more than 50 additional cycles.

In some instances, the first plurality of modified nucleotide molecules (or one or more additional pluralities of modified nucleotide molecules) may comprise a set of four different modified nucleotide molecules, where each different modified nucleotide molecule (e.g., each modified nucleotide molecule comprising a different nucleobase) is coupled to a different fluorophore.

In some instances, the first plurality of modified nucleotide molecules (or one or more additional pluralities of modified nucleotide molecules) may comprise a set of four different modified nucleotide molecules, where three of the four different modified nucleotide molecules are coupled to different fluorophores and one of the four different modified nucleotide molecules is not conjugated to a fluorophore.

In some instances, the first plurality of modified nucleotide molecules (or one or more additional pluralities of modified nucleotide molecules) may comprise a set of four different modified nucleotide molecules, where two of the four different modified nucleotides are coupled to different fluorophores, one of the four different modified nucleotide molecules is coupled to the two different fluorophores, and one of the four different modified nucleotide molecules is not conjugated to a fluorophore.

In some instances, the first plurality of modified nucleotides (or one or more additional pluralities of modified nucleotide molecules) may be selected from modified A, T, U, C, and G. In some instances, the first plurality of modified nucleotides (or one or more additional pluralities of modified nucleotide molecules) may be selected from modified A, T, C, and G.

In some instances, the first plurality of modified nucleotide molecules and at least one additional plurality of modified nucleotide molecules may comprise a same set of modified nucleotide molecules (e.g., modified nucleotides comprising the same set of nucleobases). In some instances, the first plurality of modified nucleotide molecules and at least one additional plurality of modified nucleotide molecules may comprise different sets of modified nucleotide molecules (e.g., modified nucleotides comprising different sets of nucleobases).

In some instances, the first plurality of modified nucleotide molecules and at least one additional plurality of modified nucleotide molecules may each comprise modified nucleotide molecules that do not include a 3′ reversible terminator moiety.

In some instance, the first plurality of modified nucleotide molecules and at least one additional plurality of modified nucleotide molecules may each comprise one or more modified nucleotide molecules that are coupled to a detectable label, e.g., a fluorophore. In some instances, modified nucleotide molecules that are coupled to a detectable label and that comprise a different nucleobase are coupled to a different detectable label, e.g., a different fluorophore.

In some instances, the first plurality of modified nucleotide molecules and at least one additional plurality of modified nucleotide molecules may each comprise at least one modified nucleotide molecule that is not labeled with a detectable label.

Methods for processing the series of optical signals detected over the course of performing a cyclic series of click chemistry-based base-by-base sequencing reactions to identify a nucleotide sequence are described elsewhere herein.

### III. Compositions and Kits

In some aspects, provided herein are compositions comprising any of the primers, polymerases, modified nucleotides, modified, 3′ reversibly terminated nucleotides, and/or primary probes (e.g., circular probes or circularizable probes or probe sets) described herein. Also provided herein are kits for sequencing nucleic acid molecules, including kits for sequencing and analysis of target nucleic acids in a biological sample according to any of the methods described herein.

In some instances, provided herein is a kit comprising any of the pluralities of modified nucleotides comprising a first functional group and/or modified, 3′ reversibly terminated nucleotides comprising a second functional group (or detectably-labeled versions thereof) described herein. In some instances, the kit further comprises any of the primers described herein (e.g., a priming strand comprising a modified 3′ reversibly terminated nucleotide that comprises a second functional group, or a priming strand that lacks a modified 3′ reversibly terminated nucleotide at its 3′ terminus). In some instances, provided herein is a kit further comprising any of the polymerases described herein.

In some instances, provided herein is a kit for performing click chemistry-based in situ sequencing comprising a plurality of modified nucleotide molecules as described herein, and one or more further components for performing the in situ sequencing reaction. In some instances, the one or more further components include a polymerase, a primer, modified, 3′ reversibly terminated nucleotide molecules, a support for a tissue or cell sample, or any combination thereof. In some instances, the kit further comprises any of the circular probes and/or circularizable probes or probe sets disclosed herein. In some instances, the kit comprises a polymerase for rolling circle amplification.

In some instances, provided herein is a kit for click chemistry-based flow cell sequencing comprising a plurality of a plurality of modified nucleotide molecules as described herein, and one or more further components for performing the flow cell sequencing reaction. In some instances, the one or more further components include a polymerase, a primer, modified 3′ reversibly terminated dinucleotide molecules, a flow cell, primers, adapters for sequencing library preparation, or any combination thereof.

The various components of the kit may be present in separate containers or certain compatible components may be pre-combined into a single container. In some instances, the kits further contain instructions for using the components of the kit to practice the provided methods. In some instances, sets of modified nucleotide molecules comprising a first functional group (as described elsewhere) may be provided together in a single container, such as a tube. In some instances, each of the modified nucleotide molecules comprising a first functional group may be provided in separate containers. In some instances, a first combination of modified nucleotide molecules comprising a first functional group are provided together in a first container, and a second combination of modified nucleotide molecules comprising a first functional group may be provided in a second container.

In some aspects, provided herein is a kit for sequencing a template nucleic acid molecule, comprising: a plurality of modified nucleotide molecules that each comprise a first functional group; a primer designed to hybridize to the template nucleic acid molecule, wherein the primer comprises a modified 3′ reversibly terminated nucleotide molecule that comprises a second functional group that is reactive with the first functional group; a modified 3′ reversibly terminated nucleotide that comprises the second functional group; and a polymerase. In some instances, the plurality of modified nucleotide molecules comprises a set of four modified nucleotide molecules, wherein each of the four modified nucleotide molecules comprises a different nucleobase and a different detectable feature. In some embodiments, the detectable feature is selected from: (i) a fluorophore that is different from fluorophores coupled to other modified nucleotide molecules in the set, and (ii) an absence of a fluorophore. In some instances, the plurality of modified nucleotide molecules comprises a set of four different modified nucleotide molecules, wherein each different modified nucleotide molecule is coupled to a different fluorophore. In some instances, the first plurality of modified nucleotide molecules comprises a set of four different modified nucleotide molecules, wherein three of the four different modified nucleotide molecules are coupled to different fluorophores and one of the four different modified nucleotide molecules is not conjugated to a fluorophore. In some instances, the first plurality of modified nucleotide molecules comprises a set of four different modified nucleotide molecules, wherein two of the four different modified nucleotides are coupled to different fluorophores, one of the four different modified nucleotide molecules is coupled to the two different fluorophores, and one of the four different modified nucleotide molecules is not conjugated to a fluorophore.

In some aspects, provided herein is a kit for sequencing a template nucleic acid molecule, comprising: a first plurality of modified nucleotide molecules that each comprise a first functional group; a primer designed to hybridize to the template nucleic acid molecule, wherein the primer comprises a modified 3′ reversibly terminated nucleotide that comprises a second functional group that is reactive with the first functional group; a modified 3′ reversibly terminated nucleotide that comprises the second functional group; and a polymerase. In some instances, the kit comprises an additional plurality of modified nucleotide molecules that each comprise a first functional group. In some instances, the first plurality of modified nucleotide molecules and the additional plurality of modified nucleotide molecules comprise a same set of modified nucleotide molecules. In some instances, the first plurality of modified nucleotide molecules and the additional plurality of modified nucleotide molecules comprise different sets of modified nucleotide molecules. In some instances, the first plurality of modified nucleotide molecules and the additional plurality of modified nucleotide molecules each comprise modified nucleotide molecules that do not include a 3′ reversible terminator moiety. In some instances, the first plurality of modified nucleotide molecules and the additional plurality of modified nucleotide molecules each comprise one or more modified nucleotide molecules that are coupled to a detectable label. In some instances, the first plurality of modified nucleotide molecules and the additional plurality of modified nucleotide molecules each comprise at least one modified nucleotide molecule that is not labeled with a detectable label. In some instances, the first plurality of modified nucleotides and the additional plurality of modified nucleotides are selected from modified A, T, C, and G. In some instances, the first plurality of modified nucleotides and the additional plurality of modified nucleotides are selected from modified A, T, U, C, and G. In some instances, the kit comprises at least 2, at least 5, at least 10, at least 25, at least 50, at least 75, at least 100, at least 300, at least 1,000 additional pluralities of modified nucleotide molecules that each comprise a first functional group as described herein.

In some aspects, provided herein is a kit for sequencing a template nucleic acid molecule, comprising: a first plurality of modified nucleotide molecules that each comprise a first functional group; a primer designed to hybridize to the template nucleic acid molecule, wherein the primer comprises a modified 3′ reversibly terminated nucleotide that comprises a second functional group that is reactive with the first functional group; a modified 3′ reversibly terminated nucleotide that comprises the second functional group; and a polymerase. In some embodiments, the modified 3′ reversibly terminated nucleotide is a modified 3′-O-blocked reversibly terminated nucleotide. In some embodiments, the modified 3′-O-blocked reversibly terminated nucleotide is a modified 3′-O-azidomethyl deoxynucleotide triphosphate (3′-O-azidomethyl dNTP), a modified 3′-O-allyl deoxynucleotide triphosphate (3′-O-allyl-dNTP), or a modified 3′-O-amino deoxynucleotide triphosphate (3′-O-NH₂ dNTP). In some embodiments, the modified 3′ reversibly terminated nucleotide is a modified 3′-unblocked reversibly terminated nucleotide.

In some aspects, provided herein is a kit for sequencing a template nucleic acid molecule, comprising: a first plurality of modified nucleotide molecules that each comprise a first functional group; a primer designed to hybridize to the template nucleic acid molecule, wherein the primer comprises a modified 3′ reversibly terminated nucleotide that comprises a second functional group that is reactive with the first functional group; a modified 3′ reversibly terminated nucleotide that comprises the second functional group; and a polymerase. In some embodiments, the first functional group comprises an azido group and the second functional group comprises an alkynyl group. In some embodiments, the first functional group comprises an alkynyl group and the second functional group comprises an azido group. In some embodiments, the first functional group comprises an azido group and the second functional group comprises a cyclooctynyl group. In some embodiments, the first functional group comprises a cyclooctynyl group and the second functional group comprises an azido group. In some embodiments, the first functional group comprises a tetrazine group and the second functional group comprises a dienophile group. In some embodiments, the first functional group comprises a dienophile group and the second functional group comprises a tetrazine group. In some embodiments, the first functional group comprises a thiol group and the second functional group comprises an alkynyl group. In some embodiments, the first functional group comprises an alkynyl group and the second functional group comprises a thiol group. In some embodiments, the first functional group comprises a cyano group and the second functional group comprises a 1,2-amino thiol group. In some embodiments, the first functional group comprises a 1,2-amino thiol group and the second functional group comprises a cyano group. In some embodiments, the first functional group comprises a nitrone group and the second functional group comprises a cyclooctynyl group. In some embodiments, the first functional group comprises a cyclooctynyl group and the second functional group comprises a nitrone group.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection and/or sequencing, such as barcode detection probes or detectable labels. In some embodiments, the kits optionally contain other components, for example nucleic acid primers,

### IV. Biological Samples & Sample Preparation

A sample disclosed herein can be or derived from any biological sample. The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. The biological sample can be obtained as a tissue sample, such as a tissue section, biopsy, a core biopsy, a cell pellet, a cell block, a needle aspirate, or fine needle aspirate. The sample can be a fluid sample, such as a blood sample, urine sample, or saliva sample. The sample can be a skin sample, a colon sample, a check swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Biological samples can be derived from a homogeneous culture or population of the subjects or organisms mentioned herein or alternatively from a collection of several different organisms. Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

In some instances, the biological sample may be provided on a substrate. In some instances, a substrate herein can be any support that is insoluble in aqueous liquid and which allows for positioning of biological samples, analytes, features, and/or reagents (e.g., probes) on the support. In some instances, a biological sample can be attached to a substrate. Attachment of the biological sample can be irreversible or reversible, depending upon the nature of the sample and subsequent steps in the analytical method. In certain instances, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose. In some instances, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### Preparation

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick. More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

In some instances, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than-25° C.

In some instances, the biological sample can be prepared using formalin-fixation and paraffin-embedding (FFPE), which are established methods. In some instances, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some instances, the methods provided herein comprises one or more post-fixing (also referred to as post-fixation) steps. In some instances, one or more post-fixing step is performed after contacting a sample with a polynucleotide disclosed herein, e.g., one or more probes such as a circular or padlock probe. In some instances, one or more post-fixing step is performed after a hybridization complex comprising a probe and a target is formed in a sample. In some instances, one or more post-fixing step is performed prior to a ligation reaction disclosed herein.

In some instances, a method disclosed herein comprises de-crosslinking the reversibly cross-linked biological sample. The de-crosslinking does not need to be complete. In some instances, only a portion of crosslinked molecules in the reversibly cross-linked biological sample are de-crosslinked and allowed to migrate.

In some instances, a biological sample can be permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some instances, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some instances, the biological sample can be permeabilized by any suitable methods. For example, one or more lysis reagents can be added to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes. Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

Additional reagents can be added to a biological sample to perform various functions prior to analysis of the sample. In some instances, DNase and Rnase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### Embedding

In some instances, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some instances, the hydrogel is formed such that the hydrogel is internalized within the biological sample. Biological samples can include analytes (e.g., protein, RNA, and/or DNA) embedded in a 3D matrix. In some instances, amplicons (e.g., rolling circle amplification products) derived from or associated with analytes (e.g., protein, RNA, and/or DNA) can be embedded in a 3D matrix. In some instances, a 3D matrix may comprise a network of natural molecules and/or synthetic molecules that are chemically and/or enzymatically linked, e.g., by crosslinking. In some instances, a 3D matrix may comprise a synthetic polymer. In some instances, a 3D matrix comprises a hydrogel.

In some aspects, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some instances, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some instances, the hydrogel is formed such that the hydrogel is internalized within the biological sample.

In some instances, the biological sample is immobilized in the hydrogel via cross-linking of the polymer material that forms the hydrogel. Cross-linking can be performed chemically and/or photochemically, or alternatively by any other suitable hydrogel-formation method.

In some instances, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some instances, one or more of the polynucleotide probe(s) and/or amplification product (e.g., amplicon) thereof can be modified to contain functional groups that can be used as an anchoring site to attach the polynucleotide probes and/or amplification product to a polymer matrix. In some instances, a modified probe comprising oligo dT may be used to bind to mRNA molecules of interest, followed by reversible or irreversible crosslinking of the mRNA molecules.

In some instances, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some instances, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some instances, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347 (6221): 543-548, 2015, the entire contents of which are incorporated herein by reference.

In some instances, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some instances, hydrogel formation occurs after contacting one or more second materials during formation of the substrate. Hydrogel formation can also occur within a structure (e.g., wells, ridges, projections, and/or markings) located on a substrate.

In some instances, hydrogel formation on a substrate occurs before, contemporaneously with, or after probes are provided to the sample. For example, hydrogel formation can be performed on the substrate already containing the probes.

In some instances, hydrogel formation occurs within a biological sample. In some embodiments, a biological sample (e.g., tissue section) is embedded in a hydrogel. In some instances, hydrogel subunits are infused into the biological sample, and polymerization of the hydrogel is initiated by an external or internal stimulus.

In instances in which a hydrogel is formed within a biological sample, functionalization chemistry can be used. In some instances, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some instances, hydrogel formation within a biological sample is permanent. For example, biological macromolecules can permanently adhere to the hydrogel allowing multiple rounds of interrogation. In some instances, hydrogel formation within a biological sample is reversible. In some instances, HTC reagents are added to the hydrogel before, contemporaneously with, and/or after polymerization. In some instances, a cell labeling agent is added to the hydrogel before, contemporaneously with, and/or after polymerization. In some instances, a cell-penetrating agent is added to the hydrogel before, contemporaneously with, and/or after polymerization.

In some instances, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and oligonucleotides. In some instances, optical labels are added to the hydrogel subunits before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some instances, a hydrogel-embedded sample is stored before or after clearing of hydrogel, in a medium (e.g., a mounting medium, methylcellulose, or other semi-solid mediums).

In some instances, a biological sample embedded in a matrix (e.g., a hydrogel) can be isometrically expanded. Isometric expansion methods that can be used include hydration, a preparative step in expansion microscopy, as described in, e.g., Chen et al., Science 347 (6221): 543-548, 2015 and U.S. Pat. No. 10,059,990, which are herein incorporated by reference in their entireties. Isometric expansion of the sample can increase the spatial resolution of the subsequent analysis of the sample. The increased resolution in spatial profiling can be determined by comparison of an isometrically expanded sample with a sample that has not been isometrically expanded. In some instances, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some instances, the sample is isometrically expanded to at least 2× and less than 20× of its non-expanded size.

### Staining and Immunohistochemistry (IHC)

To facilitate visualization, biological samples can be stained using a wide variety of stains and staining techniques. In some instances, for example, a sample can be stained using any number of stains and/or immunohistochemical reagents. One or more staining steps may be performed to prepare or process a biological sample for an assay described herein or may be performed during and/or after an assay. In some instances, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, SSDNA), RNA, an organelle or compartment of the cell. The sample may be contacted with one or more labeled antibodies (e.g., a primary antibody specific for the analyte of interest and a labeled secondary antibody specific for the primary antibody). In some instances, cells in the sample can be segmented using one or more images taken of the stained sample.

In some instances, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some instances, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some instances, the sample can be stained using Romanowsky stain, including Wright's stain, Jenner's stain, Can-Grunwald stain, Leishman stain, and Giemsa stain.

In some instances, biological samples can be destained. Any suitable methods of destaining or discoloring a biological sample may be utilized and generally depend on the nature of the stain(s) applied to the sample. For example, in some instances, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65 (8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### V. Analytes

A biological sample may comprise one or a plurality of analytes of interest. Methods for performing multiplexed assays to analyze two or more different analytes in a single biological sample are provided.

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

### Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

### Labeling Agents

In some instances, provided herein are methods and compositions for analyzing endogenous analytes (e.g., RNA, ssDNA, cell surface or intracellular proteins, and/or metabolites) in a sample using one or more labeling agents. In some instances, an analyte labeling agent may include an agent that interacts with an analyte (e.g., an endogenous analyte in a sample). In some instances, the labeling agents can comprise a reporter oligonucleotide that is indicative of the analyte or portion thereof interacting with the labeling agent. For example, the reporter oligonucleotide may comprise a barcode sequence that permits identification of the labeling agent. In some cases, the sample contacted by the labeling agent can be further contacted with a probe (e.g., a single-stranded probe sequence), that hybridizes to a reporter oligonucleotide of the labeling agent, in order to identify the analyte associated with the labeling agent. In some instances, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some instances, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some instances, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some instances, an analyte binding moiety may include any molecule or moiety capable of binding to an analyte (e.g., a biological analyt, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of non-limiting examples of labeling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some instances, an analyte binding moiety includes one or more antibodies or epitope-binding fragments thereof. The antibodies or epitope-binding fragments including the analyte binding moiety can specifically bind to a target analyte. In some instances, the analyte is a protein (e.g., a protein on a surface of the biological sample (e.g., a cell) or an intracellular protein). In some instances, a plurality of analyte labeling agents comprising a plurality of analyte binding moieties bind a plurality of analytes present in a biological sample. In some instances, the plurality of analytes includes a single species of analyte (e.g., a single species of polypeptide). In some instances in which the plurality of analytes includes a single species of analyte, the analyte binding moieties of the plurality of analyte labeling agents are the same. In some instances in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some instances, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the in situ detection techniques described herein.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31 (2): 708-715, which is entirely incorporated herein by reference for all purposes. Likewise, protein and peptide biotinylation techniques have been developed and are readily available. Sec, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some instances, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some instances, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

### Detectable Labels

In some instances, one or more hybridization probes or one or more nucleotides (or analogs thereof) can be labeled with distinguishing and/or detectable tags or labels. The tags may be distinguishable by means of their differences in fluorescence, Raman spectrum, charge, mass, refractive index, luminescence, length, or any other measurable property. The tag may be attached to one or more different positions on the nucleotide, so long as the fidelity of binding to the polymerase-nucleic acid complex is sufficiently maintained to enable identification of the complementary base on the template nucleic acid correctly. In some instances, the tag is attached to the nucleobase of the nucleotide. Alternatively, a tag is attached to the gamma phosphate position of the nucleotide.

Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes. The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some instances, the detectable label is bound to another moiety, for example, a nucleotide or nucleotide analog, and can include a fluorescent, a colorimetric, or a chemiluminescent label.

In some instances, a detectable label can be attached to another moiety, for example, a nucleotide or nucleotide analog. In some instances, one or more nucleotides can be labeled with a cleavable detectable tag or label. For example, the non-terminating fluorescently labeled nucleotides can include a DBCO-nucleotide conjugated to fluorescent compound with a disulfide linker. In some instances, a non-terminating fluorescently labeled nucleotide is incorporated into the strand without termination, and after imaging, the linker can be cleaved to remove fluorescent label. In some instances, a DBCO-nucleotide (e.g., 5-DBCO-PEG₄-UTP) can undergo a click reaction with the cleavable linker conjugated to a fluorescent label (e.g., cleavable linker-ATTO647N), and a disulfide group can be cleaved by tris(2-carboxyethyl) phosphine (TCEP) reduction together with 3′-O-azidomethyl-dNTP.

In some instances, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GencBLAzer™), CFP (Cyan Fluorescent Protein), CFP/YFP FRET, Chromomycin A3, CI-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DIA (4-Di-16-ASP), DID (DilC18 (5)), DIDS, DiI (DilC18 (3)), DiO (DiOC18 (3)), DiR (DilC18 (7)), Di-4 ANEPPS, Di-8 ANEPPS, DM-NERF (4.5-6.5 pH), DsRed (Red Fluorescent Protcin), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Rcd™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycocrythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycocrythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrinc Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARFR-1 (low pH), Sodium Green™, SpectrumAqua®, SpectrumGreen® #1, SpectrumGreen® #2, SpectrumOrange®, SpectrumRed®, SYTOR 11, SYTOR 13, SYTOR 17, SYTOR 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 542, ATTO 550, ATTO 565, ATTO Rho101, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. The label can emit a signal or alter a signal delivered to the label so that the presence or absence of the label can be detected. In some cases, coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl) phosphine (TCEP)) or enzymatically cleavable (e.g., via an esterase, lipase, peptidase, or protease).

### Generation of Products

In some instances, provided herein are methods and compositions for analyzing one or more products of an endogenous analyte and/or a labeling agent in a biological sample. In some instances, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some instances, a labeling agent that directly or indirectly binds to an analyte in the biological sample is analyzed. In some instances, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labeling agent that directly or indirectly binds to an analyte in the biological sample is analyzed.

### (a) Hybridization

In some instances, a hybridization product comprising the pairing of substantially complementary or complementary nucleic acid sequences within two different molecules can be analyzed. For example, hybridization of an endogenous analyte or the labeling agent (e.g., reporter oligonucleotide attached thereto) with another endogenous molecule or another labeling agent or a probe can be analyzed. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labeling agent and each probe may comprise one or more barcode sequences. Non-limiting examples of barcoded probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary.

### (b) Ligation

In some instances, a ligation product of an endogenous analyte and/or a labeling agent can be analyzed. In some instances, the ligation product is formed between two or more endogenous analytes. In some instances, the ligation product is formed between two or more labeling agents. In some instances, the ligation product is an intramolecular ligation of an endogenous analyte. In some instances, the ligation product is an intramolecular ligation product or an intermolecular ligation product, for example, the ligation product can be generated by the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labeling agent (e.g., the reporter oligonucleotide) or a product thereof.

In some instances, provided herein is a probe or probe set capable of DNA-templated ligation, such as from a cDNA molecule. Sec, e.g., U.S. Pat. No. 8,551,710, which is hereby incorporated by reference in its entirety. In some instances, provided herein is a probe or probe set capable of RNA-templated ligation. Sec, e.g., U.S. Pat. Pub. 2020/0224244 which is hereby incorporated by reference in its entirety. In some instances, the probe set is a SNAIL probe set. Sec, e.g., U.S. Pat. Pub. 20190055594, which is hereby incorporated by reference in its entirety. In some instances, provided herein is a multiplexed proximity ligation assay. See, e.g., U.S. Pat. Pub. 20140194311 which is hereby incorporated by reference in its entirety. In some instances, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some instances, a circular probe can be indirectly hybridized to the target nucleic acid. In some instances, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some instances, the ligation involves chemical ligation (e.g., click chemistry ligation). In some instances, the chemical ligation involves template dependent ligation. In some instances, the chemical ligation involves template independent ligation. In some instances, the click reaction is a template-independent reaction (see, e.g., Xiong and Seela (2011), J. Org. Chem. 76 (14): 5584-5597, incorporated by reference herein in its entirety). In some instances, the click reaction is a template-dependent reaction or template-directed reaction. In some instances, the template-dependent reaction is sensitive to base pair mismatches such that reaction rate is significantly higher for matched versus unmatched templates. In some instances, the click reaction is a nucleophilic addition template-dependent reaction. In some instances, the click reaction is a cyclopropane-tetrazine template-dependent reaction.

In some instances, the ligation involves enzymatic ligation. In some instances, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some instances, the ligase is a T4 RNA ligase. In some instances, the ligase is a Splint® ligase. In some instances, the ligase is a single stranded DNA ligase. In some instances, the ligase is a T4 DNA ligase. In some instances, the ligase is a ligase that has an DNA-splinted DNA ligase activity. In some instances, the ligase is a ligase that has an RNA-splinted DNA ligase activity.

In some instances, the ligation herein is a direct ligation. In some instances, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, i.e., separated by one or more intervening nucleotides or “gaps”. In some instances, said ends are not ligated directly to each other, but instead occurs either via the intermediacy of one or more intervening (so-called “gap” or “gap-filling” (oligo) nucleotides) or by the extension of 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo) nucleotide(s) which are complementary to a splint, padlock probe, or target nucleic acid. The gap may be a gap of 1 to 60 nucleotides or a gap of 1 to 40 nucleotides or a gap of 3 to 40 nucleotides. In specific instances, the gap may be a gap of about 1, 2, 3, 4, 5, 6, 7, 8, 9 or 10 or more nucleotides, of any integer (or range of integers) of nucleotides in between the indicated values. In some instances, the gap between said terminal regions may be filled by a gap oligonucleotide or by extending 3′ end of a polynucleotide. In some cases, ligation involves ligating the ends of the probe to at least one gap (oligo) nucleotide, such that the gap (oligo) nucleotide becomes incorporated into the resulting polynucleotide. In some instances, the ligation herein is preceded by gap filling. In other instances, the ligation herein does not require gap filling.

In some instances, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some instances, the ligation herein is a proximity ligation of ligating two (or more) nucleic acid sequences that are in proximity with each other, e.g., through enzymatic means (e.g., a ligase). In some instances, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

### (c) Primer Extension and Amplification

In some instances, a primer extension product of an analyte, a labeling agent, a probe or probe set bound to the analyte (e.g., a circularizable probe bound to genomic DNA, mRNA, or cDNA), or a probe or probe set bound to the labeling agent (e.g., a circularizable probe bound to one or more reporter oligonucleotides from the same or different labeling agents) can be analyzed.

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. A primer extension reaction generally refers to any method where two nucleic acid sequences become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some instances, a product of an endogenous analyte and/or a labeling agent is an amplification product of one or more polynucleotides, for instance, a circular probe or circularizable probe or probe set. In some instances, the amplifying is achieved by performing rolling circle amplification (RCA). In other instances, a primer that hybridizes to the circular probe or circularized probe is added and used as such for amplification. In some instances, the RCA comprises a linear RCA, a branched RCA, a dendritic RCA, or any combination thereof.

In some instances, the amplification is performed at a temperature between or between about 20° C. and about 60° C. In some instances, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some instances, upon addition of a DNA polymerase in the presence of appropriate dNTP precursors and other cofactors, a primer is elongated to produce multiple copies of the circular template. This amplification step can utilize isothermal amplification or non-isothermal amplification. In some instances, after the formation of the hybridization complex and association of the amplification probe, the hybridization complex is rolling-circle amplified to generate a cDNA nanoball (i.e., amplicon) containing multiple copies of the cDNA. Techniques for rolling circle amplification (RCA) include linear RCA, a branched RCA, a dendritic RCA, or any combination thereof. (Sec, e.g., Baner et al, Nucleic Acids Research, 26:5073-5078, 1998; Lizardi et al, Nature Genetics 19:226, 1998; Mohsen et al., Acc Chem Res. 2016 Nov. 15; 49 (11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29: el 18, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801). Non-limiting examples of polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some instances, the polymerase is phi29 DNA polymerase.

In some aspects, during the amplification step, modified nucleotides can be added to the reaction to incorporate the modified nucleotides in the amplification product (e.g., nanoball). Non-limiting examples of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some instances, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some instances, one or more of the polynucleotide probe(s) can be modified to contain functional groups that can be used as an anchoring site to attach the polynucleotide probes and/or amplification product to a polymer matrix. Non-limiting examples of modification and polymer matrix that can be employed in accordance with the provided instances comprise those described in, for example, WO 2014/163886, WO 2017/079406, US 2016/0024555, US 2018/0251833 and US 2017/0219465, which are herein incorporated by reference in their entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some instances, the provided methods involve embedding the one or more polynucleotide probe sets and/or the amplification products in the presence of hydrogel subunits to form one or more hydrogel-embedded amplification products. In some instances, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some instances, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some instances, the RCA template may comprise the target analyte, or a part thereof, where the target analyte is a nucleic acid, or it may be provided or generated as a proxy, or a marker, for the analyte. In some instances, different analytes are detected in situ in one or more cells using a RCA-based detection system, e.g., where the signal is provided by generating an RCA product from a circular RCA template which is provided or generated in the assay, and the RCA product is detected to detect the corresponding analyte. The RCA product may thus be regarded as a reporter which is detected to detect the target analyte. However, the RCA template may also be regarded as a reporter for the target analyte; the RCA product is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (e.g., a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

In some instances, a product herein includes a molecule or a complex generated in a series of reactions, e.g., hybridization, ligation, extension, replication, transcription/reverse transcription, and/or amplification (e.g., rolling circle amplification), in any suitable combination.

### VI. Fluorescence Detection and Imaging

### Fluorescence Detection

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some instances, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (Max Vision Biosciences), and/or a very intense black dye (e.g., Sudan Black, or comparable dark chromophore).

Examples of fluorescent labels and nucleotides and/or polynucleotides conjugated to such fluorescent labels comprise those described elsewhere herein and those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some instances, non-limiting examples of techniques and methods applicable to the provided embodiments comprise those described in, for example, U.S. Pat. Nos. 4,757,141, 5,151,507 and 5,091,519. In some instances, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264. In some instances, a fluorescent label comprises a signaling moiety that conveys information through the fluorescence absorption and/or emission properties of one or more molecules. Non-limiting examples of fluorescence properties comprise fluorescence intensity, fluorescence lifetime, emission spectrum characteristics and energy transfer.

### Imaging

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some instances, fluorescence microscopy is used for detection and imaging of the sample. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The fluorescence microscope can be or comprise any microscope that uses fluorescence to generate an image, whether it is a more simple set up like an epifluorescence microscope, or a more complicated design such as a confocal microscope, which uses optical sectioning to achieve better z-axis resolution of the sample to be imaged.

In some instances, confocal microscopy is used for detection and imaging of the sample. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity-so long exposures are often required. As only one point in the sample is illuminated at a time, 2D or 3D imaging requires scanning over a regular raster (i.e., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immune-stained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some instances, a method herein comprises subjecting the sample to expansion microscopy methods and techniques. Expansion allows individual targets (e.g., mRNA or RNA transcripts) which are densely packed within a cell, to be resolved spatially in a high-throughput manner. Expansion microscopy techniques are known in the art and can be performed as described in US 2016/0116384 and Chen et al., Science, 347, 543 (2015), each of which are incorporated herein by reference in their entirety. In some instances, the method does not comprise subjecting the sample to expansion microscopy. In some instances, the method does not comprise dissociating a cell from the sample such as a tissue or the cellular microenvironment. In some instances, the method does not comprise lysing the sample or cells therein. In some instances, the method docs not comprise embedding the sample or molecules from the sample in an exogenous matrix.

In some cases, analysis is performed on one or more images captured, and may comprise processing the image(s) and/or quantifying signals observed. In some instances, images of signals from different fluorescent channels and/or nucleotide incorporation cycles can be compared and analyzed. In some instances, images of signals (or absence thereof) at a particular location in a sample from different fluorescent channels and/or sequential incorporation cycles can be aligned to analyze an analyte at the location. For instance, a particular location in a sample can be tracked and signal spots from sequential incorporation cycles can be analyzed to detect a target polynucleotide sequence (e.g., a barcode sequence or subsequence thereof) in an analyte at the location. The analysis may comprise processing information of one or more cell types, one or more types of analytes, a number or level of analyte, and/or a number or level of cells detected in a particular region of the sample. In some instances, the analysis comprises detecting a sequence e.g., a barcode sequence present in an amplification product at a location in the sample. In some instances, the number of signals detected in a unit area in the biological sample is quantified. In some instances, the signals detected at a corresponding position in the biological sample in a plurality of images taken at different z positions (e.g., in the depth direction) is quantified and analyzed.

### VII. Barcode Sequences

In some instances, an analyte described herein can be associated with one or more barcode(s), e.g., at least two, three, four, five, six, seven, eight, nine, ten, or more barcodes. Barcodes can be used to spatially-resolve molecular components found in biological samples, for example, within a cell or a tissue sample. A barcode can be attached to an analyte or to another moiety or structure (e.g., a target-specific antibody) in a reversible or irreversible manner. In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some instances, a barcode includes two or more sub-barcodes (or barcode segments) that together function as a single barcode. For example, a polynucleotide barcode can include two or more polynucleotide sequences (e.g., sub-barcodes) that are contiguous or that are separated by one or more non-barcode sequences. In some instances, a barcode may comprise about 1, 2, 3, 4, 5, 6, 7, 8, 9, 10, or more than 10 sub-barcodes (or barcode segments). In some instances, each sub-barcode (or barcode segment) may comprise about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides. In some instances, each non-barcode sequence may comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some instances, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide. In any of the preceding instances, the methods provided herein can include analyzing the barcodes by performing sequential hybridization and detection with a plurality of labelled probes (e.g., detection oligos) or by performing in situ sequencing.

In some instances, e.g., in a barcode sequencing method, barcode sequences are detected for identification of other molecules including nucleic acid molecules (DNA or RNA) that are longer than the barcode sequences themselves, as opposed to direct sequencing of the longer nucleic acid molecules. In some instances, an N-mer barcode sequence can comprise up to 4˜unique sequences given a sequencing read of N bases, and a much shorter sequencing read may be required for molecular identification compared to non-barcoded sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some instances, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and U.S. Pat. Pub 20210164039, which are hereby incorporated by reference in their entirety.

### VIII. Cyclic Array Sequencing in a Flow Cell

In some instances, a method of click chemistry-based sequencing as provided herein includes “cyclic array sequencing” of amplified template nucleic acid molecules. Cyclic array flow cell sequencing methods generally involve performing multiple cycles of an enzymatic reaction on an array of spatially-separated oligonucleotide features (e.g., clonally-amplified colonies of template nucleic acid fragments tethered to a support surface, e.g., a flow cell surface). In some instances, the template nucleic acid is modified with known adapter sequence(s) comprising, e.g., amplification and/or sequencing primer binding sites, and then affixed to the support surface (e.g., the lumen surface(s) of a flow cell) in a random or patterned array by hybridization to surface-tethered complementary capture probes (complementary to adapter sequences) on the support surface, clonally amplified, and then probed using the aforementionedclick chemistry-based sequencing reaction as described herein. In some aspects, the click chemistry-based flow cell sequencing comprises massively parallel sequencing reaction, whereby each enzymatic reaction cycle is used to query only one base (the “interrogation” nucleobase) of the template nucleic acid fragment in each oligonucleotide feature, but thousands to billions of template nucleic acid molecules may be processed in parallel. Performing repeated cycles is then used to progressively identify the nucleic acid sequence of each template nucleic acid molecule based on patterns of detection of a signal or detection of an absence of a signal associated with binding of a modified nucleotide molecule to the template, as detected over the course of multiple reaction cycles. In some aspects, detection is often based on the use of labeled modified nucleotide molecules (e.g., fluorescently-labeled modified nucleotide molecules) and imaging (e.g., fluorescence imaging) of the array.

### Nucleic Acid Extraction

Nucleic acid extraction from cells or other biological samples may be performed using any of a variety of techniques known to those of skill in the art. For example, a typical DNA extraction procedure may comprise: (i) collection of a cell or tissue sample from which DNA is to be extracted, (ii) disruption of cell membranes (i.e., cell lysis) to release DNA and other cytoplasmic components, (iii) treatment of the lysed sample with a concentrated salt solution to precipitate proteins, lipids, and RNA, followed by centrifugation to separate out the precipitated proteins, lipids, and RNA, and (iv) purification of DNA from the supernatant (e.g., using spin columns or paramagnetic beads) to remove detergents, proteins, salts, or other reagents used during the cell membrane lysis step. Non-limiting examples of methods for performing nucleic acid (e.g., DNA and RNA) extraction are described in, for example, Ali et al. (2017) “Current Nucleic Acid Extraction Methods and Their Implications to Point-of-Care Diagnostics”, BioMed Research International 2017:9306564, and Dairawan et al. (2020), “The Evolution of DNA Extraction Methods”, Am J Biomed Sci & Res 8 (1): 39-45, the entire contents of each of which are incorporated herein by reference.

A variety of suitable commercial nucleic acid extraction and purification kits are consistent with the disclosure herein. Examples include, but are not limited to, the QIAamp® kits (for isolation of genomic DNA from human samples) and DNAeasy kits (for isolation of genomic DNA from animal or plant samples) from Qiagen (Germantown, Md.), or the Maxwell® and ReliaPrep™ series of kits from Promega (Madison, Wis.).

### Sequencing Library Preparation

Sequence library preparation may be performed using any of a variety of techniques known to those of skill in the art. Library preparation typically comprises performing the steps of, e.g., end repair, tailing, and ligation of adapter sequences to template nucleic acid fragments. Extracted nucleic acid molecules (e.g., DNA molecule), or fragments thereof, that are typically used as the input for sequencing library preparation often have overhangs containing single-stranded DNA (ssDNA overhangs), breaks in the phosphodiester backbone that exist on just one strand (nicks), and/or ssDNA regions internal to the duplex molecule (ssDNA gaps). End repair reactions (using, e.g., a combination of 3′ exonuclease digestion to remove 3′ overhangs and a strand displacing polymerase reaction using dNTPs to fill nicks and gaps) are used to correct these defects in order to maximize the yield for capturing and sequencing the extracted DNA, and result in the generation of blunt-ended, double-stranded DNA (dsDNA) molecules.

Tailing (e.g., A tailing) is an enzymatic method (using, e.g., a Taq DNA polymerase) for adding a non-templated nucleotide (e.g., an A nucleotide) to the 3′ end of a blunt-ended, double-stranded DNA molecule that facilitates the ligation of the adapter sequences used for sequencing.

One or more adapter sequences may then be ligated to the ends of the end-repaired and tailed template nucleic acid molecules. The adapter sequences may comprise, for example, (i) capture sequences (e.g., the Illumina p5 and p7 adapter sequences) that allow the nucleic acid molecules of the library to bind to a flow cell surface comprising complementary capture probes, (ii) amplification primer binding sites for use in performing reverse transcription and/or for generating clonally-amplified clusters on a flow cell surface, (iii) sequencing primer binding sites (e.g., the Illumina Rd1 and Rd2 sequencing primer binding site sequences) used to initiate sequencing. In addition to amplification and/or sequencing primer binding sites, in some instances the adapters may comprise a barcode sequence, e.g., a sample identification barcode sequence (such as the Illumina Index 1 and Index 2 sample identifier sequences).

Non-limiting examples of methods for performing sequencing library preparation are described in, for example, Head et al. (2014), “Library construction for next-generation sequencing: Overviews and challenges”, BioTechniques 56 (1): 61-77, and Hess et al. (2020), “Library preparation for next generation sequencing: A review of automation strategies”, Biotechnology Advances 41:107537, the entire contents of each of which are incorporated herein by reference.

### Nucleic Acid Amplification (Including Clonal Amplification)

In some instances, the disclosed methods for performing nucleic acid sequencing (e.g., in vitro and/or flow cell sequencing) may comprise performing one or more steps (e.g., 1, 2, 3, 4, 5, or more than 5) steps of nucleic acid amplification. Amplification reactions with respect to in situ based sequencing methods as described herein are discussed previously. In some instances, for example, one or more steps of nucleic acid amplification may be performed as part of sequencing library preparation and/or following sequencing library preparation. In some instances, one or more steps of nucleic acid amplification (e.g., using a solid-phase amplification technique such as bridge amplification) may be performed after the template molecules of a sequencing library have been tethered to a support surface (e.g., a flow cell surface) to generate clonally-amplified colonies of the tethered template nucleic acid fragments.

In some instances, nucleic acid amplification may be performed to amplify all of the nucleic acid molecules extracted from a biological sample (e.g., using a random primer sequence). In some instances, nucleic acid amplification may be performed to amplify a selected subset of nucleic acid molecules extracted from a biological sample (e.g., using one or more primer sequences designed to hybridize to portions of the sequences for one or more target nucleic acid molecules of interest, or to sequences adjacent thereto).

In some instances, nucleic acid amplification may be performed to amplify an entire sequencing library (e.g., using a primer sequence that hybridizes to a common amplification primer binding site in the sequencing library adapters). In some instances, nucleic acid amplification may be performed to amplify selected portions of the sequencing library (e.g., using one or more primer sequences designed to hybridize to one or more amplification primer binding sites associated with one or more identifier sequences (or barcodes) included in the sequencing library adapters).

Nucleic acid amplification may be performed using any of a variety of nucleic acid amplification techniques known to those of sill in the art, including both thermal and/or isothermal nucleic acid amplification techniques. Examples of suitable thermal nucleic acid amplification techniques include, but are not limited to, polymerase chain reaction (PCR), multiplexed PCR, nested PCR, bridge PCR, reverse transcription PCR (RT-PCR). Examples of suitable isothermal nucleic acid amplification techniques include, but are not limited to, rolling circle amplification (RCA), nucleic acid sequence-based amplification (NASBA), loop-mediated isothermal amplification (LAMP), strand displacement amplification (SDA), helicase-dependent amplification (HDA), nicking enzyme amplification reaction (NEAR), and recombinase polymerase amplification (RPA). Examples of methods for performing nucleic acid amplification are described in, for example, Gill et al. (2008), “Nucleic Acid Isothermal Amplification Technologies-A Review”, Nucleosides, Nucleotides, and Nucleic Acids 27:224-243, Fakruddin et al. (2013), “Nucleic acid amplification: Alternative method of polymerase chain reaction”, J Pharm Bioallied Sci. 5 (4): 245-252, and U.S. Pat. No. 8,143,008, the entire contents of each of which are incorporated herein by reference.

### Nucleotides

In some instances, the disclosed methods for performing nucleic acid sequencing (e.g., in situ and/or flow cell sequencing) may comprise the use of modified versions (e.g., comprising a first functional group or a second function group, as described elsewhere herein) of any of a variety of naturally-occurring nucleotides and/or functional analogs thereof (e.g., nucleotide analogs capable of hybridizing to a nucleic acid sequence in a sequence-specific/correctly base-paired manner) to probe a template nucleic acid sequence. Naturally-occurring nucleotides include deoxyribonucleotides (found in DNA) that comprise a deoxyribose sugar moiety, and ribonucleotides (found in RNA) that comprise a ribose sugar moiety. Naturally-occurring deoxyribonucleotides comprise a nucleobase (or “base”) selected from the group consisting of adenine (A), thymine (T), cytosine (C), or guanine (G). Naturally-occurring ribonucleotides comprise a nucleobase selected from the group consisting of uracil (U), adenine (A), cytosine (C), or guanine (G). In some instances, the nucleotides may be terminated (e.g., reversibly terminated). In some instances, the nucleotides may be conjugated to a detectable label, e.g., a fluorophore. In some instances, the nucleotides may be conjugated to other moieties, e.g., reactive functional groups.

### Primers (or Primer Sequences)

In some instances, the disclosed methods for performing nucleic acid sequencing (e.g., in situ and/or flow cell sequencing) can comprise the use of primer sequences that are complementary to, e.g., a subsequence (or primer binding site) that is part of an endogenous nucleic acid target sequence or a sequence (or primer binding site) that is located at or near a barcode (identifier) sequence associated with a target analyte. In some instances, a primer sequence may be designed to hybridize to a primer binding site associated with a single target analyte sequence and/or an associated target-specific barcode sequence. In some instances, a primer sequence may be designed to hybridize to a sequence (or primer binding site) that is associated with a plurality of target analyte sequences and/or associated target-specific barcode sequences (e.g., at least 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 1000, or more than 1000 target analyte sequences and/or associated target-specific barcode sequences).

### Polymerases

In some instances, the disclosed methods for performing nucleic acid sequencing (e.g., in situ and/or flow cell sequencing) may comprise performing one or more steps of nucleic acid amplification or replication using one or more polymerases. Examples of polymerases that may be used for amplification include, but are not limited to, DNA polymerases (e.g., Taq DNA polymerase), RNA polymerases, and/or reverse transcriptases.

As noted elsewhere herein, non-limiting examples of polymerases for use in rolling circle amplification (RCA) comprise DNA polymerases such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some aspects, the polymerase is phi29 DNA polymerase.

### Base-Calling

As noted elsewhere herein, the disclosed methods for performing nucleic acid sequencing (e.g., in situ and/or flow cell sequencing) may comprise inferring the sequence of a template nucleic acid molecule from a series of optical signals (e.g., fluorescence signals) detected in images acquired during a repetitive series of sequencing reaction cycles in a process referred to as “base-calling”. The interplay of sequencing chemistry, opto-fluidics hardware, optical sensors, and signal processing software utilized in sequencing platforms affects the types of errors made during sequencing (see, e.g., Lederberger et al. (2011), “Base-calling for next-generation sequencing platforms”, Brief Bioinform. 12 (5): 489-497). The characterization of errors associated with the sequencing process and implementation of chemistry-, imaging-, and/or signal processing software-based methods for minimizing sequence errors are thus important for maximizing the accuracy of sequencing results.

In four-color sequencing-by-synthesis methods, for example, a set of four images—one image for each of four detection channels corresponding to the emission wavelengths for four fluorophores used to label the reversibly terminated nucleotides—are acquired in each sequencing cycle. Processing of the images to detect fluorescence intensity signals produces an intensity quadruple for the location of each sequencing colony on a flow cell surface (or the location of each target analyte, or amplified representation thereof (e.g., an RCP) in the case of in situ sequencing), where each value represents the intensity of the fluorescence signal for the detection channels corresponding to A, C, G and T. Ideally, the channel in which the maximum intensity occurs would be the base that is “called” for a given RCP or sequencing colony (or target analyte) in a given cycle. However, the chemical processes involved in sequencing are imperfect, leading to errors in base-calling (see, e.g., Cacho, et al. (2016), “A Comparison of Base-calling Algorithms for Illumina Sequencing Technology”, Briefings in Bioinformatics 17 (5): 786-795). In some sequencing-by-synthesis (SBS) platforms, for example, sources of error may include phasing (or lagging; e.g., where the primed template nucleic acid molecules at one or more locations fail to incorporate the next base due to variation in polymerase reaction kinetics), pre-phasing (or leading; e.g., where more than one nucleotide is incorporated in a single cycle due to, e.g., impurities in the reversibly terminated nucleotides), signal decay (due to, e.g., photobleaching and/or loss of template nucleic acid during the sequencing process), and cross-talk (e.g., when two or more fluorophore emission spectra overlap, which may cause a positive correlation between signal intensities measured in the corresponding detection channels).

A variety of statistical approaches have been developed to correct for, or minimize, such errors and generate more accurate base-calls. Examples include, but are not limited to, AYB (Goldman Group, European Molecular Biology Laboratory-European Bioinformatics Institute, Cambridgeshire, UK), and Bustard (Illumina, Inc., San Diego, CA).

### Sequence Reads

The output of the base-calling process applied to optical signals detected in a series of images of a biological sample or flow cell surface acquired during a cycling sequencing process consists of a plurality of sequence reads, e.g., the nucleotide sequences determined for all or a portion of a template nucleic acid molecule (e.g., an endogenous nucleic acid analyte or a barcode sequence associated with a target analyte).

In some instances, the sequence reads generated using the disclosed methods for in situ and/or flow cell sequencing may comprise sequence reads of at least about 10, 20, 30, 40, 50, 60, 70, 80, 90, 100 or more nucleotides or base pairs of the template nucleic acid sequences. In some instances, the sequence reads generated using the disclosed methods may comprise sequence reads of at least about 100, 120, 140, 160, 180, 200, 220, 240, 260, 280, 300, 320, 340, 360, 380, 400, or more than 400 nucleotides or base pairs of the template nucleic acid sequences.

In some instances, the disclosed methods for in situ or flow cell sequencing may generate at least about 100, 200, 300, 400, 500, 600, 700, 800, 900, 1,000, or more sequencing reads per run. In some instances, the disclosed method may generate at least about 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 10⁶, 5×10⁶, 10⁷, or more than 10⁷ sequencing reads per run.

### Sequence Assembly & Alignment

In some instances, the disclosed methods for click chemistry-based in situ and/or flow cell sequencing may comprise assembly of longer template nucleic acid sequences, e.g., genome fragments or whole genomes, from a plurality of relatively short sequence reads. Sequence assembly may be performed by identifying the overlapping sequences from multiple short sequence reads to assemble longer, contiguous sections of sequence.

In some instances, the disclosed methods for in situ and/or flow cell sequencing may comprise identifying a code word corresponding to a sequence read or an assembled sequence, where the code word is one of a plurality of code words in a codebook that includes assignment of each of the plurality of code words to a target analyte of interest. The sequence read or assembled sequence may thus be used to identify a specific target analyte (based on the corresponding code word) in, e.g., a multiplexed in situ detection or sequencing assay.

In some instances, the disclosed methods for in situ and/or flow cell sequencing may comprise alignment of sequence reads and/or assembled sequences to a known reference sequence or consensus sequence (e.g., the GRCh38 human reference genome (Genome Reference Consortium)) from the same or a similar organism. Alignment to a reference sequence or consensus sequence may be used to identify gaps, errors, or variants in the assembled sequence. Any of a variety of bioinformatics software programs known to those of skill in the art may be used to assemble longer sequences from relatively short sequence reads. Examples include, but are not limited to, DBG2OLC (see, e.g., Ye et al. (2016), “DBG2OLC: Efficient Assembly of Large Genomes Using Long Erroneous Reads of the Third Generation Sequencing Technologies”, Scientific Reports 6:31900), SPAdes (see, e.g., Bankevich et al. (2012), “SPAdes: A New Genome Assembly Algorithm and Its Applications to Single-Cell Sequencing”, J. Computational Biol. 19 (5): 455-477), SparseAssembler (see, e.g., Ye et al. (2012), “Exploiting Sparseness in de novo Genome Assembly”, BMC Bioinformatics 13 (Suppl 6): S1), Fermi (see, e.g., Li (2012), “Exploring Single-Sample SNP and INDEL Calling with Whole-Genome de novo Assembly”, Bioinformatics 28 (14): 1838-1844), and String Graph Assembler (SGA) (see, e.g., Simpson et al. (2012), “Efficient de novo Assembly of Large Genomes Using Compressed Data Structures”, Genome Res. 22:549-556).

### IX. Opto-Fluidic Instrument

In some instances, the sequencing methods described herein (e.g., click chemistry-based in situ sequence or flow cell sequencing) include using instruments having integrated optics and fluidics modules (“opto-fluidic instruments” or “opto-fluidic systems”) for detecting target molecules (e.g., nucleic acids, proteins, antibodies, etc.) in biological samples (e.g., one or more cells or a tissue sample) as described herein.

In an opto-fluidic instrument, the fluidics module is configured to deliver one or more reagents (e.g., detectably-labeled and/or non-labeled nucleotide molecules, modified nucleotide molecules, reversibly terminated nucleotide molecules, modified reversibly terminated nucleotide molecules, primers, detectable-labeled probes and/or non-labeled probes, polymerases and/or other enzymes, deprotection reagents, buffers, etc.) to the biological sample (e.g., to a sample cartridge within which the biological sample is contained) or to a flow cell (e.g., within which nucleic acid molecules extracted from the biological sample have been tethered) and/or to remove spent reagents therefrom. In some instances, one or more sample preparation steps (e.g., fixing, embedding, sample clearing, and/or nucleic acid extraction (in the case that nucleic acid molecules are to be extracted and sequenced in a flow cell)) may be performed prior to the sample being placed on the instrument. In some instances, the fluidics module is configured to deliver one or more further reagents (e.g., primary probe(s) such as circular probe(s) or circularizable probe(s) or probe set(s)) and/or to remove non-specifically hybridized probe(s). In some instances, the fluidics module is configured to deliver one or more detectably labeled probes and optionally intermediate probes to detect the target analytes, or amplified representatives thereof (e.g., RCP(s)) in the biological sample. In some instances, the fluidics module is configured to deliver one or more nucleotide mixtures (e.g., mixtures of detectably-labeled and/or non-labeled modified nucleotide molecules, modified reversibly terminated nucleotide molecules, as well as primers, polymerases, deprotection reagents, etc.) to sequence, e.g., native nucleic acid sequences, barcode sequences associated with target analytes, or amplified copies thereof (e.g., barcode sequences included in RCP(s)) in the biological sample. In some instances, the fluidics module is configured to deliver one or more nucleotide mixtures (e.g., mixtures of detectably-labeled and/or non-labeled modified nucleotide molecules, modified reversibly terminated nucleotide molecules, as well as primers, polymerases, deprotection reagents, etc.) to a flow cell to sequence, e.g., native nucleic acid sequences, barcode sequences, or amplified copies thereof extracted from the biological sample.

Additionally, the optics module is configured to illuminate the biological sample (or flow cell) with light having one or more spectral emission curves (over a range of wavelengths) and subsequently capture one or more images of emitted light signals from the biological sample (or flow cell) during one or more decoding (e.g., probing or sequencing) cycles. In various instances, the captured images may be processed in real time and/or at a later time to determine the presence of the one or more target molecules in the biological sample, as well as two-dimensional and/or three-dimensional position information associated with each detected target molecule within the biological sample. In various instances, the captured images of a flow cell surface may be processed in real time and/or at a later time to determine the sequence of the one or more nucleic acid sequences (e.g., barcode sequences associated with one or more target molecules) that have been extracted from a biological sample. In some embodiment, the optics module further comprises an autofocus mechanism configured to maintain focus at a specified sample plane (e.g., a plane that is perpendicular to the optical axis of an objective lens of the optics module).

Additionally, the opto-fluidics instrument includes a sample module configured to receive (and, optionally, secure) one or more biological samples (e.g., biological samples contained with one or more sample cartridges), or to receive (and, optionally, secure) one or more flow cells. In some instances, the sample module includes an X-Y stage configured to move the biological sample (or flow cell) along an X-Y plane (e.g., perpendicular to the optical axis of an objective lens of the optics module).

In various instances, the opto-fluidic instrument is configured to analyze one or more target molecules (e.g., one or more target RNAs) in their naturally occurring place (i.e., in situ) within the biological sample. In some instances, the opto-fluidic instrument is configured to analyze one or more target RNAs in relative spatial locations within the biological sample. For example, an opto-fluidic instrument may be an in-situ analysis system used to analyze a biological sample and detect target molecules including, but not limited to, DNA, RNA, proteins, antibodies, and/or the like. In some instances, the in situ analysis system is used to detect one or more target RNAs using target-primed rolling circle amplification (RCA) according to the methods disclosed herein.

In various instances, the opto-fluidic instrument may be configured to perform in situ target molecule detection via base-by-base sequencing (e.g., by sequencing an identifier sequence such as a barcode sequence associated with a target molecule) and/or any imaging or target molecule detection technique. That is, for example, an opto-fluidic instrument may include a fluidics module that includes fluids needed for establishing the experimental conditions required for the probing or sequencing of target molecules (or associate barcode sequences) in the sample. Further, such an opto-fluidic instrument may also include a sample module configured to receive the sample, and an optics module including an imaging system for illuminating (e.g., exciting one or more fluorescent probes within the sample) and/or imaging light signals received from the probed sample. The in-situ analysis system may also include other ancillary modules configured to facilitate the operation of the opto-fluidic instrument, such as, but not limited to, cooling systems, motion calibration systems, etc.

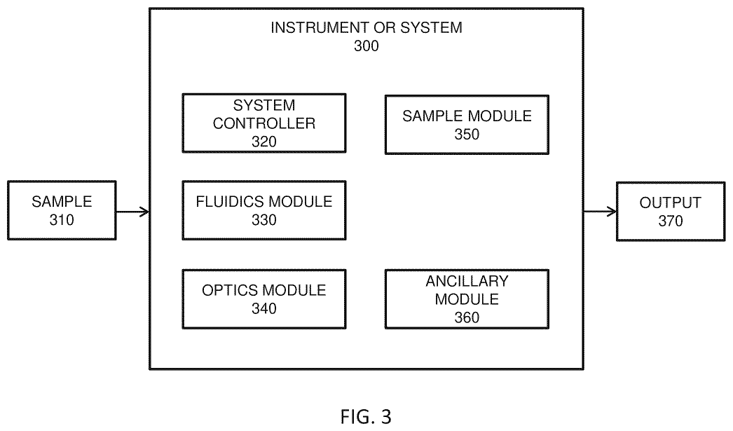

FIG. 3 shows an example workflow of analysis of a biological sample 310 (e.g., cell or tissue sample) using an opto-fluidic instrument or system 300, according to various instances. In various instances, the sample 310 can be a biological sample (e.g., a tissue) that includes molecules such as DNA, RNA, proteins, antibodies, etc. For example, the sample 310 can be a sectioned tissue that is treated to access the RNA thereof for probe (e.g., circularizable probe) hybridization and sequencing (e.g., using a sequencing primer that hybridizes to RCPs to sequence barcode sequences in the RCPs) described elsewhere herein.

In various instances, the sample 310 may be placed in the opto-fluidic instrument or system 300 for analysis and detection of the molecules in the sample 310. In various instances, the opto-fluidic instrument or system 300 can be a system configured to facilitate the experimental conditions conducive for the detection of the target molecules. For example, the opto-fluidic instrument or system 300 can include a fluidics module 330, an optics module 340, a sample module 350, and an ancillary module 360, and these modules may be operated by a system controller 320 to create the experimental conditions for hybridization probe-based detection and/or base-by-base sequencing of nucleic acid molecules in the sample 310, as well as to facilitate the imaging of the sample (e.g., by an imaging system of the optics module 340). In various instances, the various modules of the opto-fluidic instrument or system 300 may be separate components in communication with each other, or at least some of them may be integrated together.

In various instances, the sample module 350 may be configured to receive the sample 310 into the opto-fluidic instrument or system 300. For instance, the sample module 360 may include a sample interface module (SIM) that is configured to receive a sample device (e.g., cassette) onto which the sample 310 can be deposited. That is, the sample 310 may be placed in the opto-fluidic instrument or system 300 by depositing the sample 310 (e.g., the sectioned tissue) on a sample device that is then inserted into the SIM of the sample module 350. In some instances, the sample module 350 may also include an X-Y stage onto which the SIM is mounted. The X-Y stage may be configured to move the SIM mounted thereon (e.g., and as such the sample device containing the sample 310 inserted therein) in perpendicular directions along the two-dimensional (2D) plane of the opto-fluidic instrument or system 300.

The experimental conditions that are conducive for the detection of the molecules in the sample 310 may depend on the target molecule detection technique that is employed by the opto-fluidic instrument or system 300. For example, in various instances, the opto-fluidic instrument or system 300 can be a system that is configured to detect molecules (e.g., by detecting hybridization probes that hybridize to nucleic molecules (e.g., barcode sequences) and/or by nucleotides incorporated into extending sequencing primers using an identifier sequence as a template) in the sample 310.

In various instances, the fluidics module 330 may include one or more components that may be used for storing the reagents, as well as for transporting said reagents to and from the sample device containing the sample 310. For example, the fluidics module 330 may include reservoirs configured to store the reagents, as well as a waste container configured for collecting the reagents (e.g., and other waste) after use by the opto-fluidic instrument or system 300 to analyze and detect the molecules of the sample 310. Further, the fluidics module 330 may also include pumps, tubes, pipettes, etc., that are configured to facilitate the transport of the reagent to the sample device (e.g., and as such the sample 310). For instance, the fluidics module 330 may include pumps (“reagent pumps”) that are configured to pump washing/stripping reagents to the sample device for use in washing/stripping the sample 310 (e.g., as well as other washing functions such as washing an objective lens of the imaging system of the optics module 340).

In various instances, the ancillary module 360 can be a cooling system of the opto-fluidic instrument or system 300, and the cooling system may include a network of coolant-carrying tubes that are configured to transport coolants to various modules of the opto-fluidic instrument or system 300 for regulating the temperatures thereof. In such cases, the fluidics module 330 may include coolant reservoirs for storing the coolants and pumps (e.g., “coolant pumps”) for generating a pressure differential, thereby forcing the coolants to flow from the reservoirs to the various modules of the opto-fluidic instrument or system 300 via the coolant-carrying tubes. In some instances, the fluidics module 330 may include returning coolant reservoirs that may be configured to receive and store returning coolants, e.g., heated coolants flowing back into the returning coolant reservoirs after absorbing heat discharged by the various modules of the opto-fluidic instrument or system 300. In such cases, the fluidics module 330 may also include cooling fans that are configured to force air (e.g., cool and/or ambient air) into the returning coolant reservoirs to cool the heated coolants stored therein. In some instance, the fluidics module 330 may also include cooling fans that are configured to force air directly into a component of the opto-fluidic instrument or system 300 so as to cool said component. For example, the fluidics module 330 may include cooling fans that are configured to direct cool or ambient air into the system controller 320 to cool the same.

As discussed above, the opto-fluidic instrument or system 300 may include an optics module 340 which include the various optical components of the opto-fluidic instrument or system 300, such as but not limited to a camera, an illumination module (e.g., LEDs), an objective lens, and/or the like. The optics module 340 may include a fluorescence imaging system that is configured to image the fluorescence emitted by the detectably labeled nucleotides are incorporated in extending sequencing primers in the sample 310 after the detectable labels are excited by light from the illumination module of the optics module 340.

In some instances, the optics module 340 may also include an optical frame onto which the camera, the illumination module, and/or the X-Y stage of the sample module 350 may be mounted.

In various instances, the system controller 320 may be configured to control the operations of the opto-fluidic instrument or system 300 (e.g., and the operations of one or more modules thereof). In some instances, the system controller 320 may take various forms, including a processor, a single computer (or computer system), or multiple computers in communication with each other. In various instances, the system controller 320 may be communicatively coupled with data storage, set of input devices, display system, or a combination thereof. In some cases, some or all of these components may be considered to be part of or otherwise integrated with the system controller 320, may be separate components in communication with each other, or may be integrated together. In other examples, the system controller 320 can be, or may be in communication with, a cloud computing platform.

In various instances, the opto-fluidic instrument or system 300 may analyze the sample 310 and may generate the output 370 that includes indications of the presence of the target molecules in the sample 310. For instance, with respect to instances discussed above where the opto-fluidic instrument or system 300 employs a sequencing technique for detecting molecules, the opto-fluidic instrument or system 300 may cause the sample 310 to undergo successive sequencing cycles, where during the same sequencing cycle the sample is imaged to detect signals associated with nucleotide binding and/or incorporation events at some locations in the sample 310, as well as to detect an absence of signals at other locations in the sample. In such cases, the output 370 may include a series of optical signals (e.g., a code word) specific to each identifier sequence (e.g., a barcode sequence), which allow the identification of the target molecules.

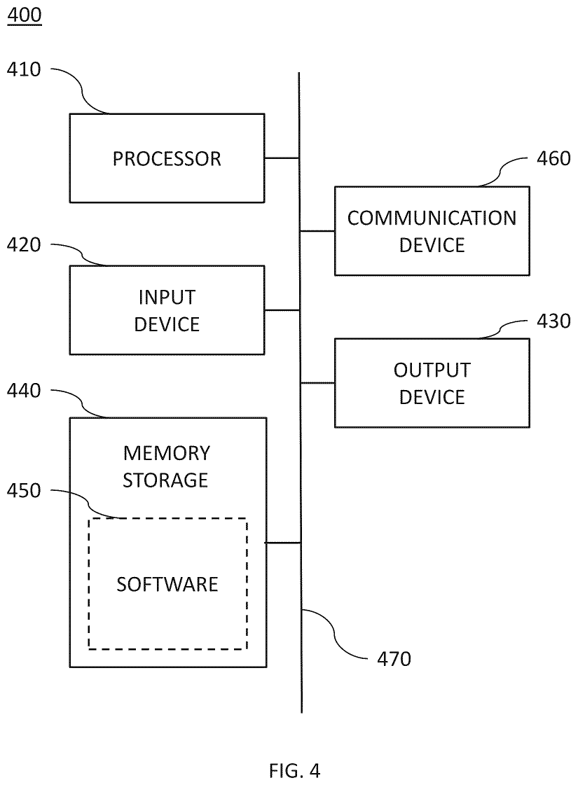

FIG. 4 illustrates an example of a computing device or system in accordance with one or more examples of the disclosure. Device 400 can be a host computer connected to a network. Device 400 can be a client computer or a server. As shown in FIG. 4, device 400 can be any suitable type of microprocessor-based device, such as a personal computer, workstation, server, or handheld computing device (portable electronic device), such as a phone or tablet. The device can include, for example, one or more of processor 410, input device 420, output device 430, memory/storage 440, and communication device 460. Input device 420 and output device 430 can generally correspond to those described above, and they can either be connectable or integrated with the computer.

Input device 420 can be any suitable device that provides input, such as a touch screen, keyboard or keypad, mouse, or voice-recognition device. Output device 430 can be any suitable device that provides output, such as a touch screen, haptics device, or speaker.

Storage 440 can be any suitable device that provides storage, such as an electrical, magnetic, or optical memory including a RAM, cache, hard drive, or removable storage disk. Communication device 460 can include any suitable device capable of transmitting and receiving signals over a network, such as a network interface chip or device. The components of the computer can be connected in any suitable manner, such as via a physical bus 470 or wirelessly.

Software 450, which can be stored in memory/storage 440 and executed by processor 410, can include, for example, the programming that embodies the functionality of the present disclosure (e.g., as embodied in the methods and systems described above). Software 450 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 440, that can contain or store programming for use by or in connection with an instruction execution system, apparatus, or device.

Software 450 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

Device 400 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, Tl or T3 lines, cable networks, DSL, or telephone lines.

Device 400 can implement any operating system suitable for operating on the network. Software 450 can be written in any suitable programming language, such as C, C++, Java, or Python. In various implementations, application software embodying the functionality of the present disclosure can be deployed in different configurations, such as in a client/server arrangement or through a web browser as a web-based application or web service, for example.

### XII. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” and “nucleic acid molecule,” used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide.

The term “about” as used herein refers to the usual error range for the respective value readily known to the skilled person in this technical field. Reference to “about” a value or parameter herein comprises (and describes) embodiments that are directed to that value or parameter per sc.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The following examples are included for illustrative purposes only and are not intended to limit the scope of the present disclosure.

### Example 1: Sequencing Using Click Chemistry Bioconjugation In Situ

This example provides a workflow for using a click chemistry reaction between a modified 3′ reversibly terminated nucleotide and a fluorescently labeled modified nucleotide molecule to sequence a template nucleic acid molecule (e.g., DNA or RNA) in a tissue section. Use of click chemistry to retain the fluorescently labeled modified nucleotide molecule may provide certain advantages such as stable retention of a fluorescent read-out.

A tissue sample is obtained and cryosectioned onto a glass slide for processing. The tissue is fixed by incubating in 3.7% paraformaldehyde (PFA). To prepare for probe hybridization, a wash buffer is added to the tissue sample. The washed tissue sample is then contacted with a circularizable probe comprising a template nucleic acid molecule and a barcode sequence. The barcode sequence identifies a target analyte within the tissue sample. The circularizable probe is allowed to hybridize to the target analyte. The tissue sample is then contacted with a ligation reaction mix including ligase, and the circularizable probe is ligated to form a circular template for rolling circle amplification (RCA). The tissue sample is then incubated with an RCA mixture containing a Phi29 DNA polymerase and dNTP for RCA of the circularized probes. From this amplification, the RCA product (e.g., RCP) comprises the template nucleic acid molecule and a barcode sequence.

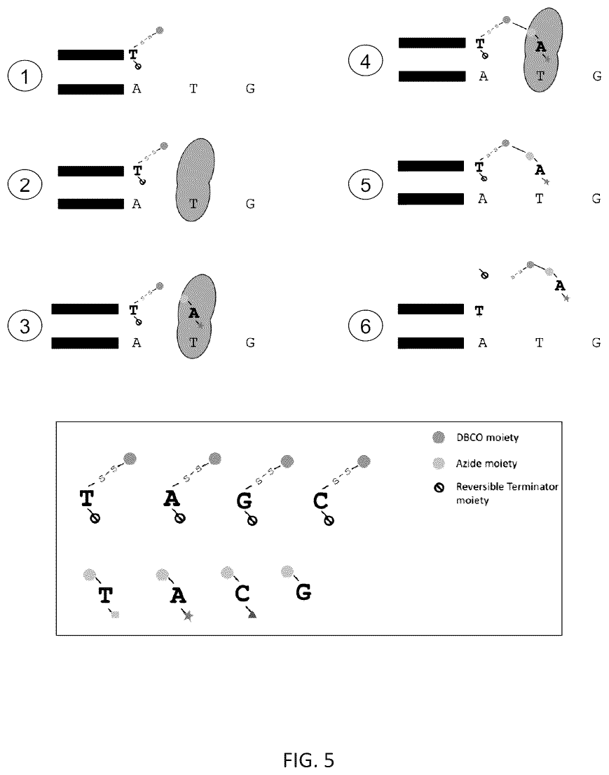

A non-limiting example of the disclosed sequencing methods is illustrated schematically in FIG. 5. In the case of in situ sequencing, the tissue sample is washed and then contacted with a primer comprising a modified 3′ reversibly terminated nucleotide, wherein the nucleotide comprises, e.g., a dibenzocyclooctynyl (DBCO) group attached to the base of the nucleotide via a disulfide linker. For the purines, the DBCO modification is attached to the N7 position of the base indirectly via a disulfide linker, and for pyrimidines the modification is attached to the C5 position of the base indirectly via a disulfide linker. The primer is allowed to hybridize to the template nucleic acid molecule in the RCP (step 1 in FIG. 5, where the upper strand is the primer and the lower strand is the template nucleic acid molecule). The tissue sample is washed and then contacted (in one or more steps) with a polymerase (step 2 in FIG. 5) and a first plurality of modified nucleotides that each include an azido group. The first plurality of modified nucleotides includes four sets of modified nucleotide molecules, where each of the four sets of modified nucleotide molecules has a different nucleobase type and a different label from the other four sets (three different fluorophores and one “dark” label). Each of the modified nucleotides of the plurality is modified with an azide group attached at 5′ position of the sugar. A modified nucleotide molecule of the first plurality and the polymerase are allowed to form a complex with the primer hybridized to the template nucleic acid molecule (step 3 in FIG. 5). The DBCO group of the modified 3′ reversibly terminated nucleotide is allowed to react with the azido group of a modified nucleotide molecule of the first plurality that is complementary to a nucleotide residue in the template nucleic acid molecule to form a click chemistry product (step 4 in FIG. 5). The tissue sample is then washed to remove polymerase and unbound modified nucleotide molecules of the first plurality (step 5 in FIG. 5).

Fluorescence imaging is used to detect a signal associated with the presence of the modified nucleotide molecule of the first plurality covalently coupled to the modified 3′ reversibly terminated nucleotide of the primer. Images for each of a plurality of detection channels configured to detect signals arising from labels (e.g., fluorescent dyes) conjugated to nucleotide molecules present in the complex are acquired in each cycle of a multicycle sequencing run.

The tissue sample is contacted with a buffer comprising dithiothreitol (DTT) and a deprotection reagent. The reducing agent is allowed to disrupt the disulfide linker between the modified 3′ reversibly terminated nucleotide and the click chemistry product to release the modified nucleotide molecule of the first plurality. The primer bound to the template nucleic acid molecule is deprotected (step 6 in FIG. 5). The tissue sample is washed to remove the released modified nucleotide molecule of the first plurality. The tissue sample is contacted with an additional modified 3′ reversibly terminated nucleotide comprising a DBCO group attached to the nucleotide via a disulfide linker. The additional modified 3′ reversibly terminated nucleotide is complementary to the identified nucleotide in the template nucleic acid molecule. An extension reaction is performed to incorporate the additional modified 3′ reversibly terminated nucleotide into the extended priming strand. After primer extension, the entire cycle as described above is repeated until the barcode is sequenced.

### Example 2: Sequencing Using Click Chemistry Bioconjugation in a Flow Cell

This example provides a workflow for using a click chemistry reaction between a modified 3′ reversibly terminated nucleotide and a modified nucleotide molecule to sequence a template nucleic acid molecule (e.g., DNA or RNA) immobilized on a flow cell surface. Use of click chemistry to retain the modified nucleotide molecule may provide certain advantages such as stable retention of a fluorescent read-out.

Again, the sequencing process is illustrated schematically in FIG. 5. The template nucleic acid molecules to be sequenced are attached to the surface of a flow cell at discrete locations (e.g., where each location comprises multiple clonally-amplified copies of a single template nucleic acid molecule created using bridge amplification or a similar technique). A primer that does not comprise a modified 3′ reversibly terminated nucleotide is introduced to the flow cell. The primer is allowed to hybridize to an immobilized template nucleic acid molecule. A modified 3′ reversibly terminated nucleotide that is complementary to a nucleotide in the template nucleic acid molecule is introduced to the flow cell, and an extension reaction is performed to incorporate the modified 3′ reversibly terminated nucleotide into the priming strand (step 1 in FIG. 5, where the upper strand is the primer and the lower strand is the template nucleic acid molecule). The modified 3′ reversibly terminated nucleotide comprises, e.g., a trans-cyclooctene group attached to the nucleotide via a disulfide linker.

The flow cell is washed and then contacted (in one or more steps) with a polymerase (step 2 in FIG. 5) and a first plurality of modified nucleotides that each comprise a tetrazine group. The first plurality of modified nucleotide molecules comprises, e.g., a set of four different modified nucleotide molecules, and where three of the four different modified nucleotide molecules are coupled to different fluorophores and one of the four different modified nucleotide molecules is not conjugated to a fluorophore. A modified nucleotide molecule of the first plurality and the polymerase are allowed to form a complex with the primer hybridized to the template nucleic acid molecule (step 3 in FIG. 5). The trans-cyclooctene group of the modified 3′ reversibly terminated nucleotide is allowed to react with the tetrazine group of the modified nucleotide molecule of the first plurality to form a click chemistry product (step 4 in FIG. 5). The flow cell is washed to remove polymerase and unbound modified nucleotide molecules of the first plurality (step 5 in FIG. 5).

Fluorescence imaging is used to detect a signal or absence of a signal associated with the presence of the modified nucleotide molecule of the first plurality covalently coupled to the modified 3′ reversibly terminated nucleotide of the primer. Images for each of a plurality of detection channels configured to detect signals arising from labels (e.g., fluorescent dyes) conjugated to nucleotide molecules present in the complex are acquired in each cycle of a multicycle sequencing run.

The flow cell is contacted with a buffer comprising dithiothreitol (DTT) and a deprotection reagent. The reducing agent is allowed to disrupt the disulfide linker between the modified 3′ reversibly terminated nucleotide and the click chemistry product to release the modified nucleotide molecule of the first plurality. The primer bound to the template nucleic acid molecule is deprotected (step 6 in FIG. 5). The flow cell is washed to remove the released modified nucleotide molecule. The flow cell is contacted with an additional modified 3′ reversibly terminated nucleotide comprising a trans-cyclooctene group attached to the nucleotide via a disulfide linker. The additional modified 3′ reversibly terminated nucleotide is complementary to the previously identified nucleotide in the template nucleic acid molecule. An extension reaction is performed to incorporate the additional modified 3′ reversibly terminated nucleotide into the extended priming strand. After primer extension, the entire cycle as described above is repeated until the template nucleic acid molecule is sequenced.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method for sequencing a template nucleic acid molecule comprising:
a) contacting a priming strand bound to the template nucleic acid molecule with (i) a polymerase and (ii) a first plurality of modified nucleotide molecules that each have a first functional group, to form a complex comprising a 3′ terminus of the priming strand, the template nucleic acid molecule, the polymerase, and a first modified nucleotide molecule of the first plurality of modified nucleotide molecules, wherein the priming strand comprises a modified 3′ reversibly terminated nucleotide that comprises a second functional group, and wherein the first modified nucleotide molecule is not incorporated into a sugar-phosphate backbone of the priming strand;
b) performing a coupling reaction between the first functional group and the second functional group to covalently couple the first modified nucleotide molecule included in the complex to the modified 3′ reversibly terminated nucleotide; and
c) detecting a presence of the first modified nucleotide molecule coupled to the modified 3′ reversibly terminated nucleotide.

2. (canceled)

3. The method of claim 1, wherein the coupling reaction comprises a click chemistry reaction.

4. The method of claim 1, wherein the first functional group of each of the first plurality of modified nucleotides is attached to the modified nucleotide using a first linker.

5.-6. (canceled)

7. The method of claim 4, wherein the first linker comprises a cleavable linker.

8. (canceled)

9. The method of claim 1, wherein the second functional group is attached to the modified 3′ reversibly terminated nucleotide using a second linker.

10. The method of claim 9, wherein the second linker comprises a cleavable linker.

11.-12. (canceled)

13. The method of claim 1, wherein the first functional group is attached directly or indirectly to the nucleobase of each of the plurality of modified nucleotide molecules.

14.-15. (canceled)

16. The method of claim 1, wherein the first functional group is attached directly or indirectly to the sugar of each of the plurality of modified nucleotide molecules.

17. The method of claim 1, wherein the second functional group is attached directly or indirectly to the nucleobase of the modified 3′ reversibly terminated nucleotide.

18.-19. (canceled)

20. The method of claim 1, wherein the second functional group is attached directly or indirectly to the sugar of the modified 3′ reversibly terminated nucleotide.

21. The method of claim 1, wherein the first functional group and second functional group are selected from a list of click reactive functional group pairs comprising azido/alkynyl groups, alkynyl/azido groups, azido/dibenzocyclooctynyl (DBCO) groups, dibenzocyclooctynyl (DBCO)/azido groups, azido/cyclooctynyl groups, cyclooctynyl/azido groups, tetrazine/dienophile groups, dienophile/tetrazine groups, thiol/alkynyl groups, alkynyl/thiol groups, cyano/1,2-amino thiol groups, 1,2-amino thiol/cyano groups, nitrone/cyclooctynyl groups, cyclooctynyl/nitrone groups, or any combination thereof.

22.-40. (canceled)

41. The method of claim 1, further comprising:
d) performing a cleavage reaction to remove the covalently coupled modified nucleotide molecule from the modified 3′ reversibly terminated nucleotide;
e) performing a deprotection reaction to deprotect the modified 3′ reversibly terminated nucleotide; and
f) performing an extension reaction to incorporate a modified 3′ reversibly terminated nucleotide that comprises the second functional group and that is complementary to the identified nucleotide in the template nucleic acid molecule into an extended priming strand.

42.-44. (canceled)

45. The method of claim 41, wherein the second functional group is attached indirectly to 3′ reversibly terminated molecule via a cleavable linker, and d) performing the cleavage reaction comprises contacting the complex with a reagent capable of cleaving the cleavable linker.

46. (canceled)

47. The method of claim 41, further comprising repeating steps (a)-(f) for at least one additional cycle using at least one additional plurality of modified nucleotide molecules that comprise the first functional group to detect a presence of a modified nucleotide molecule coupled to the modified 3′ reversibly terminated nucleotide and identify at least one additional complementary nucleotide in the template nucleic acid molecule.

48. (canceled)

49. The method of claim 1, further comprising:
prior to performing a first contacting step in (a), hybridizing a primer that does not comprise a modified 3′ reversibly terminated nucleotide at its 3′ end to a primer binding site in the template nucleic acid molecule; and
performing an extension reaction to incorporate a modified 3′ reversibly terminated nucleotide that comprises the second functional group and that is complementary to a nucleotide in the template nucleic acid molecule into an extended primer strand to generate the priming strand.

50.-55. (canceled)

56. The method of claim 1, wherein detecting a presence of a modified nucleotide molecule in the complex comprises detecting a signal associated with a detectable label coupled to the modified nucleotide molecule.

57. (canceled)

58. The method of claim 1, wherein the polymerase is not labeled with a detectable label.

59. (canceled)

60. The method of claim 1, wherein the first plurality of modified nucleotide molecules comprises four different sets of modified nucleotide molecules, and wherein each modified nucleotide molecule of a set is coupled to a different fluorophore from those of the other sets.

61.-68. (canceled)

69. The method of claim 1, wherein the template nucleic acid molecule comprises a target analyte nucleic acid molecule.

70. The method of claim 1, wherein the template nucleic acid molecule comprises a barcode sequence associated with a target analyte.

71. The method of claim 70, further comprising hybridizing a circularizable probe to the target analyte or to a labeling agent bound to the target analyte and ligating the circularizable probe to form a circularized probe, wherein the method further comprises performing rolling circle amplification of the circularized probe to generate the template nucleic acid molecule.

72.-73. (canceled)

74. The method of claim 1, wherein the template nucleic acid molecule to be sequenced is attached to a solid support.

75. The method of claim 74, wherein the solid support comprises a sequencing flow cell.

76. The method of claim 1, wherein the template nucleic acid molecule is sequenced in situ in a cell sample or tissue sample.

77. (canceled)

78. A kit for sequencing a template nucleic acid molecule comprising:
a plurality of modified nucleotide molecules that each comprise a first functional group;
a primer designed to hybridize to the template nucleic acid molecule; and
a modified 3′ reversibly terminated nucleotide that comprises the second functional group.

79. The kit of claim 78, wherein the plurality of modified nucleotide molecules comprises four sets of modified nucleotide molecules, wherein each of the four sets of modified nucleotide molecules comprises a different nucleobase and a different detectable feature, wherein the detectable feature is selected from: (i) a fluorophore that is different from fluorophores coupled to other modified nucleotide molecules in the set, and (ii) optionally, an absence of a fluorophore.

80.-81. (canceled)

82. The method of claim 1, wherein the method further comprises identifying a complementary nucleotide in the template nucleic acid molecule based on the presence of the first modified nucleotide molecule.

83. A method for sequencing a template nucleic acid molecule comprising:
providing:
i) one or more reagents comprising a polymerase and a first plurality of modified nucleotide molecules, each comprising a first functional group, and
ii) a priming strand bound to the template nucleic acid molecule, wherein the priming strand comprises a modified 3′reversibly terminated nucleotide comprising a second functional group;
a) contacting the priming strand bound to the template nucleic acid molecule with the one or more reagents, to form a complex comprising a 3′ terminus of the priming strand, the template nucleic acid molecule, the polymerase, and a first modified nucleotide molecule of the first plurality of modified nucleotide molecules, wherein the first modified nucleotide molecule is not incorporated into a sugar-phosphate backbone of the priming strand;
b) performing a coupling reaction between the first functional group and the second functional group to covalently couple the first modified nucleotide molecule included in the complex to the modified 3′ reversibly terminated nucleotide; and
c) detecting a presence of the first modified nucleotide molecule coupled to the modified 3′ reversibly terminated nucleotide.

84. The method of claim 83, wherein the method further comprises identifying a complementary nucleotide in the template nucleic acid molecule based on the presence of the first modified nucleotide molecule.

85. The kit of claim 78, further comprising a polymerase.


SEQUENCE LISTING (6 sequences)

SEQ ID NO: 1 - protein, 306 aa, Thermus aquaticus
GMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLXAFLERLEFGSLLHEFGLLESPKXLXEAPWPPPERAFVP

SEQ ID NO: 2 - protein, 575 aa, Bacillus virus phi29 [sequence omitted]

SEQ ID NO: 3 - protein, 775 aa, Thermococcus sp.
  source 1..775: note = strain 9oN-7
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFDIETLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 4 - protein, 928 aa, synthetic construct [sequence omitted]

SEQ ID NO: 5 - protein, 560 aa, Moloney murine leukemia virus
AFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLGRLEDLKSKTLGDLVREAEKIFNKRETPEEREERIRRETEEKEERRRTVDEQKEKERDRRRHREMSKLLATVVIGQEQDRQEGERKRPQLDKDQCAYCKEKGHWAKDCPKKPRGPRGPRPQTSLLTLGDXGGQGQDPPPEPRITLKVGGQPVTFLVDTGAQHSVLTQNPGPLSDKSAWVQGATGGKRYRWTTDRKVHLATGKVTHSFLHVPDCPYPLLGRDLLTKLKAQIHFEGSGAQVVGPMGQPLQVLTLNIEDEYRLHETSKEPDVSLGFTWLSDFPQAWAESGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFR

SEQ ID NO: 6 - protein, 259 aa, Human immunodeficiency virus 1
PISPIEPVPVKLKPGMDGPKVKQWPLTEEKIKALVEICTEMEKEGKISKIGPENPYNTPVFAIKKKDSTRWRKLVDFRELNKRTQDFWEVQLGIPHPAGLKKKRSVTVLDVGDAYFSVPLDKEFRKYTAFTIPSINNETPGIRYQYNVLPQGWKGSPAIFQSSMTKILEPFRKQNPDIVIYQYMDDLYVGSDLEIGQHRTKIEELRQHLLKWGFTTPDKKHQKEPPFLWMGYEHHPDKWTVQPIVLPEKDSWTVNDIQK